(19) United States
(12) Patent Application Publication
(10) Pub. No.: US 2026/0062763 A1
(43) Pub. Date: Mar. 5, 2026

(54) DIGITAL PCR ASSAY FOR THE DETECTION OF ENTEROHEMORRHAGIC ESCHERICHIA COLI

(71) Applicant: The Florida State University Research Foundation, Inc., Tallahassee, FL (US)

(72) Inventor: Prashant Singh, Tallahassee, FL (US)

(21) Appl. No.: 19/320,249

(22) Filed: Sep. 5, 2025

Related U.S. Application Data
(60) Provisional application No. 63/691,097, filed on Sep. 5, 2024.

(51) Int. Cl. C12Q 1/689; C12Q 1/6806; C12Q 1/6816; C12Q 1/686; G01N 33/12

(52) U.S. Cl. CPC C12Q 1/689; C12Q 1/6806; C12Q 1/6816; C12Q 1/686; G01N 33/12; C12Q 2600/16

(57) ABSTRACT

The present disclosure relates primer pairs, probes, kits, and methods of use thereof to detect virulent strains of Shiga toxin-producing Escherichia coli (E. coli) (STEC).

### RELATED APPLICATION

This U.S. utility application claims priority to, and the benefit of, U.S. Provisional Patent Application No. 63/691,097, filed Sep. 5, 2024, entitled “DIGITAL PCR ASSAY FOR THE DETECTION OF ENTEROHEMORRHAGIC ESCHERICHIA COLI,” which is incorporated by reference herein in its entirety.

### REFERENCE TO SEQUENCE LISTING

The sequence listing submitted on Sep. 5, 2025, as an .XML file entitled “10850-117US1_ST26” created on Sep. 5, 2025, and having a file size of 26,909 bytes is hereby incorporated by reference pursuant to 37 C.F.R. § 1.52(e)(5).

### FIELD

The present disclosure relates primer pairs, probes, kits, and methods of use thereof to detect virulent strains of Shiga toxin-producing Escherichia coli (E. coli) (STEC).

### BACKGROUND

Enterohemorrhagic Escherichia coli (EHEC) is one of the biggest threats to the food industry. EHEC strains are responsible for multiple beef and fresh produce-related recalls. The USDA FSIS has a zero-tolerance policy for the top-seven EHEC serogroups. Therefore, the beef industry must test for the presence of pathogens before product release. The USDA-FSIS and beef industry are the primary users of EHEC detection methods. They rely on multiple real-time PCR assays. The problem with the real-time PCR assay is the false-positive results. The USDA has reported 81-100% false-positive results for these assays. The assay picks signals from other bacteriophages and Gram-negative bacteria present in the food samples. Therefore, there is an urgent need for an assay that can detect the presence of target virulence genes in one cell.

### SUMMARY

The present disclosure provides primer pairs, probes, assays comprising said primer pairs and probes, and methods of use thereof to detect a Shiga toxin-producing Escherichia coli (E. coli) (STEC) serotype.

In some aspects, disclosed herein is a partition-based PCR assay comprising a primer pair selected from SEQ ID NO: 1 and SEQ ID NO: 2, SEQ ID NO: 5 and SEQ ID NO: 6, SEQ ID NO: 8 and SEQ ID NO: 9, SEQ ID NO: 11 and SEQ ID NO: 12, or a combination thereof; and a probe selected from SEQ ID NO: 3, SEQ ID NO: 4, SEQ ID NO: 7, SEQ ID NO: 10, SEQ ID NO: 13, or a combination thereof, wherein the probe comprises a modified nucleic acid and at least one detectable label.

In some embodiments, the assay comprises two primer pairs selected from (a) SEQ ID NO: 1 and SEQ ID NO: 2, and SEQ ID NO: 5 and SEQ ID NO: 6; (b) SEQ ID NO: 1 and SEQ ID NO: 2, and SEQ ID NO: 8 and SEQ ID NO: 9; (c) SEQ ID NO: 1 and SEQ ID NO: 2, and SEQ ID NO: 11 and SEQ ID NO: 12; (d) SEQ ID NO: 5 and SEQ ID NO: 6, and SEQ ID NO: 8 and SEQ ID NO: 9; (e) SEQ ID NO: 5 and SEQ ID NO: 6, and SEQ ID NO: 11 and SEQ ID NO: 12; or (f) SEQ ID NO: 8 and SEQ ID NO: 9, and SEQ ID NO: 11 and SEQ ID NO: 12.

In some embodiments, the assay comprises three primer pairs selected from (a) SEQ ID NO: 1 and SEQ ID NO: 2, SEQ ID NO: 5 and SEQ ID NO: 6, and SEQ ID NO: 8 and SEQ ID NO: 9; (b) SEQ ID NO: 1 and SEQ ID NO: 2, SEQ ID NO: 5 and SEQ ID NO: 6, and SEQ ID NO: 11 and SEQ ID NO: 12; (c) SEQ ID NO: 1 and SEQ ID NO: 2, SEQ ID NO: 8 and SEQ ID NO: 9, and SEQ ID NO: 11 and SEQ ID NO: 12; or (d) SEQ ID NO: 5 and SEQ ID NO: 6, SEQ ID NO: 8 and SEQ ID NO: 9, and SEQ ID NO: 11 and SEQ ID NO: 12.

In some embodiments, the assay comprises SEQ ID NO: 3, SEQ ID NO: 4, SEQ ID NO: 7, SEQ ID NO: 10, SEQ ID NO: 13, or a combination thereof. In some embodiments, the assay comprises SEQ ID NO: 3, SEQ ID NO: 4, SEQ ID NO: 7, SEQ ID NO: 10, and SEQ ID NO: 13.

In some embodiments, the detectable label comprises a fluorophore, a quencher, or a combination thereof. In some embodiments, the modified nucleic acid includes, but is not limited to a locked nucleic acid (LNA). In some embodiments, the kit detects one or more virulent genes selected from stx, eae, and a O157:H7 serotype-specific gene. In some embodiments, the kits detects one or more Shiga toxin-producing Escherichia coli (E. coli) (STEC) serogroup. In some embodiments, the kit detects strains from one or more STEC serogroup, but is not limited to O157, O26, O45, O103, O111, O121, O145, O22, O55, O64, O86, O147, or a variant thereof. In some embodiments, the kit detects the one or more virulent genes directly from a single E. coli cell.

In some aspects, disclosed herein is a method of detecting one or more Shiga toxin-producing E. coli (STEC) serotypes in a food product, the method comprising (a) enriching a bacterial cell within the food product; (b) isolating the bacterial cell from the food product; (c) exposing the bacterial cell to a digital PCR component comprising at least one primer pair and at least one probe; and (d) performing a partition-based digital PCR, wherein a target nucleic acid within the bacterial cell is hybridized to at least one primer pair and at least one probe, wherein the at least one primer pair comprises SEQ ID NO: 1 and SEQ ID NO: 2, SEQ ID NO: 5 and SEQ ID NO: 6, SEQ ID NO: 8 and SEQ ID NO: 9, SEQ ID NO: 11 and SEQ ID NO: 12, or a combination thereof; and the at least one probe comprises SEQ ID NO: 3, SEQ ID NO: 4, SEQ ID NO: 7, SEQ ID NO: 10, SEQ ID NO: 13, or a combination thereof, wherein the probe comprises a modified nucleic acid and a detectable label, wherein at least one virulent gene is detected in the bacterial cell, and wherein the bacterial cell is identified as a STEC.

In some embodiments, the one or more STEC strains include, but are not limited to O157, O26, O45, O103, O111, O121, O145, O22, O55, O64, O86, O147, or a variant thereof. In some embodiments, at least one or more targets virulent gene comprises stx, eae, and a O157:H7 serotype specific gene. In some embodiments, the food product is enriched for no more than 8 hours. In some embodiments, the method discriminates between a virulent STEC gene and an avirulent STEC strains. In some embodiments, the modified nucleic acid comprises a locked nucleic acid (LNA). In some embodiments, the food product comprises beef. In some embodiments, the food product comprises spinach. In some embodiments, the food product comprises lettuce.

In some aspects, disclosed herein is a probe comprising at least 90% sequence identity to SEQ ID NO: 10 or at least 90% sequence identity to SEQ ID NO: 13. In some embodiments, the probe comprises SEQ ID NO: 10 or SEQ ID NO: 13.

### BRIEF DESCRIPTION OF FIGURES

The accompanying figures, which are incorporated in and constitute a part of this specification, illustrate several aspects described below.

FIG. 1 shows the overview of the partition-based digital PCR assay.

FIG. 2 shows the 2D scatter plot of beef inoculated with a avirulent E. coli strain (Strain1: eae positive).

FIG. 3 shows the 2D scatter plot of beef inoculated with two E. coli avirulent strain (Strain 1: eae positive, Strain 2 stx positive). Even when the beef was inoculated with two avirulent strains, the assay did not show any partition with double-positive for the stx and eae genes.

FIG. 4 shows the 2D scatter plot of beef inoculated with a virulent E. coli strain (eae and stx positive). The samples showed double-positive in the top right quadrant, showing the presence of cells with stx and eae genes.

FIG. 5 shows a 1D scatter plot for the detection of E. coli O157:H7 from beef swabs inoculated with O157:H7 pathogenic strains listed in Table 3.

FIG. 6 shows the 2D graph comparing partitions positive for both stx and eae in E. coli O157:H7 OK_1.

FIG. 7 shows the 2D graph comparing partitions positive for both stx and eae in E. coli O157:H7 86_24.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used herein, “STEC” or “Shiga toxin-producing Escherichia coli” refers to a group of E. coli strains with the ability to produce Shiga toxin via the expression of stx1 and stx2 genes. STECs are broadly divided into E. coli serotype O157 and non-O157 serogroups. Specifically discussed herein are the O157, O26, O45, O103, O111, O121, O145, O22, O55, O64, O86, and O147.strains. In some other parts of the world, they are also referred to as Enterohemorrhagic Escherichia coli (EHEC).

As used herein, “multiplex” refers to refers to the use of PCR to amplify several different DNA targets (genes) simultaneously in a single assay or reaction. Multiplexing can amplify nucleic acid samples, such as genomic DNA, cDNA, RNA, etc., using multiple primers and any necessary reagents or components in a thermal cycler.

As used herein, “enrichment” refers to conditions favoring the growth of a particular microorganism. For example, in one embodiment, a method of the present disclosure may benefit from an enrichment step whereby bacterial cells or a solution obtained by homogenizing a biological sample and containing one or more target bacterial cells or species are placed in an enrichment medium to allow for the growth of the target bacterial species or strains for the purposes of detection of the bacterial cells or species.

As used herein, the term “subject,” “patient,” or “organism” includes humans and mammals (e.g., mice, rats, pigs, cats, dogs, and horses). Typical subjects for which methods of the present invention may be applied will be mammals, such as humans. A wide variety of subjects will be suitable for veterinary, diagnostic, research, or food safety applications, e.g., humans; livestock such as cattle, sheep, goats, cows, swine, and the like; poultry such as chickens, ducks, geese, turkeys, and the like; and domesticated animals, particularly pets such as dogs and cats. The term “living subject” refers to a subject as noted above or another organism that is alive.

As used herein, the term “culture media” or “media” refers to liquid, semi-solid, or solid media used to support bacterial cell growth in a non-native environment. Further, by culture media is meant a sterile solution that is capable of sustaining and/or promoting the division or survival of such cells. Suitable culture media are known to one of skill in the art, as discussed herein. The media components may be obtained from suppliers other than those identified herein and may be optimized for use by those of skill in the art according to their requirements. Culture media components are well known to one of skill in the art and concentrations and/or components may be altered as desired or needed.

In certain embodiments, sequences of the disclosure, including primer sequences, target sequences and internal amplification control (IAC) sequences may be identical to the sequences provided here in or comprise less than 100% sequence identity to the sequences provided herein. For instance, primer sequences, target sequences or IAC sequences of the present invention may comprise 90% identity to the sequences provided herein.

The terms “identical” or “percent identity,” in the context of two or more nucleic acids or sequences, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site found at ncbi.nlm.nih.gov/BLAST/ or the like). Such sequences are then referred to as “substantially identical.” This definition also refers to, or applies to, the compliment of a particular sequence. The definition may also include sequences that have deletions, additions, and/or substitutions. To compensate for gene sequence diversity and to target multiple gene variants of the same genes, degenerated primer pairs (1-2 bases or approximately 5-10% alterations) are allowed. The sequence can comprise or consist of those sequences disclosed herein.

As used herein, the term “nucleic acid” refers to a single or double-stranded polymer of deoxyribonucleotide bases or ribonucleotide bases read from the 5′ to the 3′ end, which may include genomic DNA, target sequences, primer sequences, or the like. In accordance with the invention, a “nucleic acid” may refer to any DNA or nucleic acid to be used in an assay as described herein, which may be isolated or extracted from a biological sample. The term “nucleotide sequence” or “nucleic acid sequence” refers to both the sense and antisense strands of a nucleic acid as either individual single strands or in the duplex. The terms “nucleic acid segment,” “nucleotide sequence segment,” or more generally, “segment,” will be understood by those in the art as a functional term that includes genomic sequences, target sequences, operon sequences, and smaller engineered nucleotide sequences that express or may be adapted to express, proteins, polypeptides or peptides. The nomenclature used herein is that required by Title 37 of the United States Code of Federal Regulations § 1.822 and set forth in the tables in WIPO Standard ST.25 (1998), Appendix 2, Tables 1 and 3.

As used herein, a “modified nucleic acid” refers to nucleic acids, such as for example DNA or RNA, that have been chemically altered to enhance stability, improve binding to target molecules, and regulate gene expression. Such modifications to nucleic acids can involve changes to the phosphate backbone, the sugar unit (i.e., ribose sugars), or the nitrogenous base of the nucleic acid, thus introducing extra chemical groups to create enhanced properties and functions. Non limiting examples of modified nucleic acids include locked nucleic acids (LNAs), 5-mehtylcytosine (5mC), pseudouridine, dihydrouridine, 7-methylguanosine (m7G), peptide nucleic acids (PNAs), and morpholino nucleic acids (PMO).

As used herein, the term “locked nucleic acid (LNA)”, also referred to as “bridged nucleic acid”, refers to a modified nucleic analog where a methylene bridge between the 2′-O and 4′ carbon atoms of the ribose sugar “locks” the sugar rings into a confirmation ideal for complementary binding. The structural rigidity of the LNA increases the LNA's binding affinity to DNA and RNA, resulting in higher thermal stability (i.e., melting temperatures). It should be noted that LNAs can be incorporated into oligonucleotides (such as primers and probes) to enhance their stability, increase nuclease resistance, and increased specificity to target sequences.

The term “gene” refers to components that comprise bacterial DNA or RNA, cDNA, artificial bacterial DNA polynucleotide, or other DNA that encodes a bacterial peptide, bacterial polypeptide, bacterial protein, or bacterial RNA transcript molecule, introns and/or exons where appropriate, and the genetic elements that may flank the coding sequence that are involved in the regulation of expression, such as, promoter regions, 5′ leader regions, 3′ untranslated region that may exist as native genes or transgenes in a bacterial genome. The gene or a fragment thereof can be subjected to polynucleotide sequencing methods that determines the order of the nucleotides that comprise the gene. Polynucleotides as described herein may be complementary to all or a portion of a bacterial gene sequence, including a promoter, coding sequence, 5′ untranslated region, and 3′ untranslated region. Nucleotides may be referred to by their commonly accepted single-letter codes.

The term “recombinant” when used with reference, e.g., to a cell, or nucleic acid indicates that the cell or nucleic acid has been modified by the introduction, by natural or artificial means, of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. In some embodiments, recombinant sequences may also include nucleic acids, proteins, or recombinant genomes, such as bacterial genomes. In certain embodiments, a “recombinant” bacterium or cell may refer to a bacterial cell into which a stx gene or nucleic acid has been inserted, for example by a lambdoid phage, conferring the ability of the bacterial cell to produce shiga toxin.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular electrode is disclosed and discussed and a number of modifications that can be made to the electrode are discussed, specifically contemplated is each and every combination and permutation of the electrode and the modifications that are possible unless specifically indicated to the contrary. Thus, if a class of electrodes A, B, and C are disclosed as well as a class of electrodes D, E, and F and an example of a combination electrode, or, for example, a combination electrode comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

### Digital PCR Assays and Method of Use

The present disclosure provides primer pairs, probes, assays comprising said primer pairs and probes, and methods of use thereof to detect E. coli O157:H7 and other strains of Shiga toxin-producing Escherichia coli (E. coli) (STEC) strains.

STECs are zoonotic pathogens found in the intestinal tract and feces of beef cattle and ruminants and thus can be introduced into food products of animal origin during slaughter. Beef products contaminated with animal feces have been associated with STEC infections in humans. These pathogens have also been reported to contaminate milk, cheese, and other dairy products. STEC infections lead to a variety of illnesses with varying severity, including diarrhea, hemorrhagic colitis (bloody diarrhea), hemolytic uremic syndrome (kidney failure), and death resulting in STEC strains causing human illness are included as notifiable pathogens in the Nationally Notifiable Diseases Surveillance System in 2000.

STECs are broadly divided into E. coli serotype O157 and non-O157 serogroups. In the last decade, the non-O157 serogroup has emerged as a major food-borne pathogen of concern worldwide, responsible for 63%, 74%, 82%, and 80% of the total STEC infections in Canada, Denmark, Germany, and the Netherlands, respectively. To date, a large number of STEC serogroups have been identified, but not all are pathogenic to humans. The frequency of infections of STEC serogroups is variable, with six non-O157 STEC serotypes being most commonly reported: O26 (26%), O103 (22%), O111 (19%), O121 (6%), O45 (5%), and O145 (4%), leading to their classification by the USDA as adulterants (zero tolerance) in non-intact raw beef products.

In accordance with this invention, virulent strains of O157, O26, O45, O103, O111, O121, O145, O22, O55, O64, O86, O147, and other STEC strains can be specifically and reliably detected in a biological sample, including but not limited to beef, fresh produce, and dairy products. Using the assays and methods described herein, these STECs may be specifically and reliably detected in a biological sample at low concentrations and in minimal time, thus enabling rapid and low-cost simultaneous detection of multiple pathogenic bacteria. Also, the assays and methods described herein are capable of discriminating between virulent STEC strains and avirulent STEC strains.

In some aspects, disclosed herein is a partition-based PCR assay comprising a primer pair selected from SEQ ID NO: 1 and SEQ ID NO: 2, SEQ ID NO: 5 and SEQ ID NO: 6, SEQ ID NO: 8 and SEQ ID NO: 9, SEQ ID NO: 11 and SEQ ID NO: 12, or a combination thereof; and a probe selected from SEQ ID NO: 3, SEQ ID NO: 4, SEQ ID NO: 7, SEQ ID NO: 10, SEQ ID NO: 13, or a combination thereof, wherein the probe comprises a modified nucleic acid and at least one detectable label.

In some aspects, disclosed herein is a probe comprising at least 70% sequence identity to SEQ ID NO: 10 or at least 70% sequence identity to SEQ ID NO: 13.

In some embodiments, the probe comprises 70%, 75%, 80%, 85%, 90%, 95%, 99%, or 100% sequence identity to SEQ ID NO: 10. In some embodiments, the probe comprises 70%, 75%, 80%, 85%, 90%, 95%, 99%, or 100% sequence identity to SEQ ID NO: 13. In some embodiments, the probe comprises SEQ ID NO: 10. In some embodiments, the probe comprises SEQ ID NO: 13.

In some embodiments, the probe comprises a modified nucleic acid and at least one detectable label. In some embodiments, the probe of any preceding aspect can have 1, 2, 3, 4, 5, or more detectable labels. In some embodiments, the least one detectable label comprises a fluorescent dye, a quencher, or a combination thereof.

In some embodiments, the assay comprises two primer pairs selected from (a) SEQ ID NO: 1 and SEQ ID NO: 2, and SEQ ID NO: 5 and SEQ ID NO: 6; (b) SEQ ID NO: 1 and SEQ ID NO: 2, and SEQ ID NO: 8 and SEQ ID NO: 9; (c) SEQ ID NO: 1 and SEQ ID NO: 2, and SEQ ID NO: 11 and SEQ ID NO: 12; (d) SEQ ID NO: 5 and SEQ ID NO: 6, and SEQ ID NO: 8 and SEQ ID NO: 9; (e) SEQ ID NO: 5 and SEQ ID NO: 6, and SEQ ID NO: 11 and SEQ ID NO: 12; or (f) SEQ ID NO: 8 and SEQ ID NO: 9, and SEQ ID NO: 11 and SEQ ID NO: 12.

In some embodiments, the assay comprises three primer pairs selected from (a) SEQ ID NO: 1 and SEQ ID NO: 2, SEQ ID NO: 5 and SEQ ID NO: 6, and SEQ ID NO: 8 and SEQ ID NO: 9; (b) SEQ ID NO: 1 and SEQ ID NO: 2, SEQ ID NO: 5 and SEQ ID NO: 6, and SEQ ID NO: 11 and SEQ ID NO: 12; (c) SEQ ID NO: 1 and SEQ ID NO: 2, SEQ ID NO: 8 and SEQ ID NO: 9, and SEQ ID NO: 11 and SEQ ID NO: 12; or (d) SEQ ID NO: 5 and SEQ ID NO: 6, SEQ ID NO: 8 and SEQ ID NO: 9, and SEQ ID NO: 11 and SEQ ID NO: 12.

In some embodiments, the assay comprises SEQ ID NO: 3, SEQ ID NO: 4, SEQ ID NO: 7, SEQ ID NO: 10, SEQ ID NO: 13, or a combination thereof. In some embodiments, the assay comprises SEQ ID NO: 3, SEQ ID NO: 4, SEQ ID NO: 7, SEQ ID NO: 10, and SEQ ID NO: 13. In some embodiments, the primer concentration for O157:H7 ranges from 0.1 to 5 μL. In some embodiments, the primer concentration for O157:H7 comprises 0.1 μL, 0.2 μL, 0.3 μL, 0.4 μL, 0.5 μL, 0.6 μL, 0.7 μL, 0.8 μL, 0.9 μL, 1 μL, 1.5 μL, 2 μL, 2.5 μL, 3 μL, 3.5 μL, 4 μL, 4.5 μL, or 5 μL. In some embodiments, the probe concentration for O157:H7 ranges from 0.5 to 2 μL. In some embodiments, the probe comprises 0.5 μL, 0.6 μL, 0.7 μL, 0.8 μL, 0.9 μL, 1 μL, 1.5 μL, or 2 μL. In some embodiments, the ground beef swab pulls a dilution gradient for O157:H7 ranges from 0.5 to 2500. In some embodiments, the ground beef swab pulls dilution gradient for O157:H7 comprises 0.5 μL, 0.6, 0.7, 0.8, 0.9, 1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805,806, 807, 808, 809, 810,811, 812, 813, 814, 815,816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500

In some embodiments, the kit detects one or more virulent genes selected from stx1, stx2, eae, and a O157:H7 serotype-specific gene.

STECs produce Shiga toxins via the expression of stx1 and stx2 genes, which are transferred to a bacterial cell by lambdoid phages that integrate into the bacterial chromosome. A total of 10 known stx subtypes are presently known. The stx1 and stx2 are further divided into multiple subtypes, in which stx1 has three subtypes (stx1a, stx1c, and stx1d), and stx2 has seven subtypes (stx2a, stx2b, stx2c, stx2d, stx2e, stx2f, and stx2g). Although commercial kits are presently available for identification of Shiga toxin genes, the high genetic diversity of the stx subtypes results in subtypes that may not necessarily be detected by the various assays (Feng et al., Appl Environ Microbiol 77:6699-6702, 2011; Margot et al., J Food Protection 76:871-873, 2013) or these assays are prone to false-positive test results due to presence of stx gene in the bacteriophage.

In some embodiments, the kits detects a Shiga toxin-producing Escherichia coli (E. coli) (STEC) strain including, but not limited to O157, O26, O45, O103, O111, O121, O145, O22, O55, O64, O86, O147, or a variant thereof.

In some aspects, disclosed herein is a method of detecting one or more Shiga toxin-producing E. coli (STEC) serotypes in a food product, the method comprising (a) enriching a bacterial cell within the food product; (b) isolating the bacterial cell from the food product; (c) exposing the bacterial cell to a digital PCR component comprising at least one primer pair and at least one probe; and (d) performing a partition-based digital PCR, wherein a target nucleic acid within the bacterial cell is hybridized to at least one primer pair and at least one probe, wherein the at least one primer pair comprises SEQ ID NO: 1 and SEQ ID NO: 2, SEQ ID NO: 5 and SEQ ID NO: 6, SEQ ID NO: 8 and SEQ ID NO: 9, SEQ ID NO: 11 and SEQ ID NO: 12, or a combination thereof; and the at least one probe comprises SEQ ID NO: 3, SEQ ID NO: 4, SEQ ID NO: 7, SEQ ID NO: 10, SEQ ID NO: 13, or a combination thereof, wherein the probe comprises a modified nucleic acid and a detectable label, wherein at least one virulent gene is detected in the bacterial cell, and wherein the bacterial cell is identified as a STEC.

In some embodiments, the method of detecting one or more STEC serotypes in a food product comprises using the assay of any preceding aspect. In some embodiment, the food product is enriched for no more than 8 hours. In some embodiments, the food product is enriched for 6 to 8 hours, depending on the food matrices. In some embodiments, the food product is enriched for 0.5, 1, 1.5, 2, 2.5, 3, 3.5, 4, 4.5, 5, 5.5, 6, 6.5, 7, 7.5 or 8 hours. In some embodiments, the method discriminates between a virulent STEC gene and an avirulent STEC gene. It should be understood that the enrichment time will vary depending on the food matrices. Non-limiting examples of the variation in enrichment time includes beef samples requiring eight hours of enrichment and spinach samples requiring a 6 hour enrichment.

In some embodiments, the method is suitable for testing a multitude of biological samples, for example food products. In some embodiments, a biological sample may be meat such as beef, beef stew meat, beef trimmings, chicken, turkey, dairy, water or the like. A biological sample may also include produce such as various vegetables and fruits, such as alfalfa sprouts, spinach, lettuce, or juices from vegetable or fruits such as apple cider. As used herein, a “biological sample” or “sample” may also include clinical samples such as blood and blood parts including, but not limited to serum, plasma, platelets, or red blood cells; sputum, mucosa, tissue, cultured cells, including primary cultures, explants, and transformed cells; biological fluids, stool, and urine. A biological sample may also include sections of tissues such as biopsy and autopsy samples, and frozen sections taken for histologic purposes. A biological sample may be obtained from a eukaryotic organism, for example a mammal, including humans, cows, pigs, chickens, turkeys, ducks, geese, dogs, goats, and the like. Any tissue appropriate for use in accordance with the invention may be used, for instance, skin, brain, spinal cord, adrenals, pectoral muscle, lung, heart, liver, crop, duodenum, small intestine, large intestine, kidney, spleen, pancreas, adrenal gland, bone marrow, lumbosacral spinal cord, or blood. In some embodiments, the food product comprises beef. In some embodiments, the food product comprises spinach. In some embodiments, the food product comprises lettuce.

In some embodiments, the method of any preceding aspect requires 1, 1.5, 2, 2.5, 3, 3.5, 4, 4.5, 5, 5.5, 6, 6.5, 7, 7.5 or 8 hours of enriching a biological sample of any preceding aspect comprising the E. coli cell. In some embodiments, the method of any preceding aspect may comprise the steps of: i) enriching a bacterial concentration in a test sample by incubating the sample aerobically at approximately 42° C., for instance 37° C., 38° C., 39° C., 40° C., 41° C., 42° C., 43° C., 44° C., or 45° C. in an enrichment media such as described herein; ii) isolating DNA from the enriched sample; iii) isolating the bacterial cells by centrifugation and running the assay directly on the cells without any DNA isolation, and iv) detecting sample DNA using the specific primer sets as described herein.

During the sample enrichment step, a biological sample such as a food sample or other clinical sample, may be collected and diluted in a buffer or media such as water, saline, brain heart infusion broth (BHI), tryptic soy broth (TSB), modified tryptic soy broth (mTSB) or sterile Buffered Peptone Water (BPW), among others. Media useful for culture or enrichment of STECs, Salmonella, or other food pathogens in food samples would be known by one of skill in the art. Exemplary media in accordance with the invention may include, but are not limited to, BHI, TSB, mTSB and buffered peptone water (BPW) broth. In some embodiments, a sample as described herein may be diluted at any stage in a desired buffer or solution, for example 1:10, 1:9, 1:8, 1:7, 1:6, 1:5, 1:4, 1:3, 1:2, or 1:1.

Antibiotics may be used in the enrichment of STECs in order to provide a selective advantage for pathogens to grow among any other bacteria present in a food or environmental sample. The use of antibiotics in enrichment broth may hinder the growth of other existing bacteria and simultaneously promote the selective growth of pathogens to be detected in the assay of the present invention. Selection of suitable antibiotics may ensure that the growth of the selected pathogen is not inhibited, thereby ensuring that the sample enrichment time is not lengthened unnecessarily. In some embodiments, antibiotics may be added to a sample or medium such as an enrichment medium or a culture medium. For example, VCC supplement, containing vancomycin, which deters the growth of Gram-positive bacteria, cefixime, which suppresses the growth of Proteus spp., and/or cefsulodin, which inhibits Pseudomonas spp.; novobiocin, acriflavine, penicillin, streptomycin, chloramphenicol, gentamycin, and the like. Antimycotic compounds may include, but are not limited to Fungizone or other suitable compound. Any of these compounds may be used alone or in combination where appropriate. Any suitable concentration of antibiotic or antimycotic may be used, for example 10 mg/L, 9 mg/L, 8 mg/L, 7 mg/L, etc. In other embodiments, a diluted sample may be homogenized using an appropriate device, such as a Stomacher, for a short time period (such as 2 minutes) in order to release attached cells. The homogenized samples may be incubated aerobically, such as at 42° C., or other temperature suitable for a bacterial strain, for anywhere from 4 to 12 hours to allow for enrichment (recovery and growth of target bacterial species). For example, samples may be incubated for 1 hour, 2 hours, 3 hours, 4 hours, 5 hours, or 6 hours.

In some embodiments, the method of any preceding aspect does no require DNA isolation from E. coli cells. Thus, enabling detection of presence of virulence genes withing the intact cells after enrichment.

### Amplification of DNA

Methods such as polymerase chain reaction (PCR and RT-PCR) and ligase chain reaction (LCR) may be used with the kit to amplify nucleic acid sequences.

In some embodiments, the primers are not labeled, and the amplicons may be visualized, detected, and/or analyzed by real-time PCR using a intercalating dye based approach following their melting temperature, for example by generation of melt curve assays or plots or a dual-labeled probe based approach (i.e., TaqMan, Molecular beacon, Sunrise probe). In some embodiments, an amplicon may be visualized according to size, e.g., using agarose gel electrophoresis. In some embodiments, ethidium bromide staining of the PCR amplicons following size resolution allows visualization of the different size amplicons. Such an approach may be referred to as end-point PCR. Conventional end-point PCR, while suitable for amplification and detection of a target DNA or sequence, may require extensive sample enrichment time due to the higher copy number of target DNA molecules needed for detection. This translates to a higher number of target cells, which, in turn, translates to longer enrichment times. In some embodiments, the primers of the invention may be radiolabeled, or labeled by any suitable means (e.g., using a non-radioactive fluorescent tag), to allow for rapid visualization of amplicons of different sizes following an amplification reaction without any additional labeling step or visualization step.

In accordance with the present disclosure, a PCR assay as described herein may be multiplexed in order to combine multiple reactions into a single assay. For example, a multiplex assay may enable amplification of multiple target sequences using a number of PCR primer pairs, such as one or more primers set forth in the Examples. One of skill in the art will understand that the reaction conditions for each individual reaction in a multiplex assay will necessarily be similar in order to achieve efficient amplification of each target. Optimization or other testing of each individual primer pair may be necessary. For the development of a multiplex PCR assay such as described herein, a large number of primer-pairs has to be tested for each target in order to determine the optimum primer that will produce the best result. Out of multiple PCR primers that work for a particular multiplex assay, a final set of primer pairs for a multiplex assay may be selected based on specific criteria, including, but not limited to, (1) fluoresce value of positive partition; (2) higher PCR amplification efficiency; (3) amplicon size; and (4) performance in multiplex assay.

In some embodiments, a bacterial species such as a STEC species or serogroups as described herein may be detected based on the level of a particular RNA or DNA in a biological sample. Any of the primers described herein and set forth as SEQ ID NOs:1, 2, 5, 6, 8, 9, 10, 11, 12, or 13 may be used for detection, diagnosis, and determination of the presence of such a bacterial species. An amplified nucleotide may then be detected and distinguished for other sequences using techniques described herein.

A PCR assay may include a number of reagents and components, including a master mix and nucleic acid dye or intercalating agent. In some embodiments, an exemplary PCR master mix may contain template genomic material, such as DNA, PCR primers, salts such as MgCl₂, a polymerase enzyme, and deoxyribonucleotides. One of skill in the art will be able to identify useful components of a master mix in accordance with the present invention.

During real-time PCR detection, PCR may be performed in any reaction volume, such as 10 μL, 45 μL, 50 μL. Reactions may be performed singly, in duplicate, or in triplicate. PCR thermal cycling conditions are well known in the art and vary based on a number of factors. As described herein, an exemplary two-step amplification protocol may include, for example, an initial denaturation at 94° C. for 10 min; 40 cycles of 94° C. for 30 s, 60° C. for 45 s. Any thermal cycling program may be designed as appropriate for use with the particular primers for detection of particular bacterial targets as would be understood by one of skill in the art.

Test samples or assays as described herein may be compared to a control or reference sample, such as a positive control, in order to accurately determine the presence and/or amount of a particular pathogen such as a STEC or pathogenic or antibiotic resistant bacterial species. In addition, a reaction control may be used, such as an IAC, in order to avoid false negative results and thereby increase the reliability of an assay. Use of an IAC in a reaction provides assurance that a negative result for a target is truly a negative result rather than due to a problem or break-down in the reaction. Because the signal for the IAC should always be generated, even when the target signal is not generated (i.e., the target organism or DNA is not present in the sample), this would indicate that a negative target signal is indeed a negative result. An IAC may be useful in diagnostic assays because food matrices may harbor inhibitory components that may interfere with PCR amplification, leading to false negative results.

Short oligonucleotides such as an IAC molecule as described herein may be amplified at a much higher amplification efficiency (>100%) and thus may be preferentially amplified in a multiplex PCR reaction. To overcome this issue, an IAC molecule may be added to a multiplex reaction at the lowest possible concentration (10-20 fg), facilitating preferential amplification of the desired target DNA.

### Modification of Nucleic Acids

Any number of methods well known to those skilled in the art can be used to isolate and manipulate a DNA molecule. For example, as previously described, PCR technology may be used to amplify a particular starting DNA molecule and/or to produce variants of the starting DNA molecule. DNA molecules, or fragments thereof, can also be obtained by any techniques known in the art, including directly synthesizing a fragment by chemical means. Thus, all or a portion of a nucleic acid as described herein may be synthesized.

As used herein, the term “complementary nucleic acids” refers to two nucleic acid molecules that are capable of specifically hybridizing to one another, wherein the two molecules are capable of forming an anti-parallel, double-stranded nucleic acid structure. In this regard, a nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are known in the art and described by Sambrook, et al. (1989), and by Haymes et al. (1985).

Departures from complete complementarity are permissible, as long as the capacity of the molecules to form a double-stranded structure remains. Thus, in order for a nucleic acid molecule or a fragment of the nucleic acid molecule to serve as a primer or probe, such a molecule or fragment need only be sufficiently complementary in sequence to be able to form a stable double-stranded structure under the particular solvent and salt concentrations employed.

Appropriate stringency conditions that promote DNA hybridization are well known to one of skill in the art and may include, for example, 6× sodium chloride/sodium citrate (SSC) at about 45° C., followed by a wash of 2×SSC at 50° C. The salt concentration in the wash step may be selected from a low stringency of approximately 2×SSC at 50° C. to a high stringency of about 0.2×SSC at 50° C. The temperature in the wash step may be increased from low stringency conditions at room temperature, about 22° C., to high stringency conditions at about 65° C. The temperature and/or salt conditions may be varied as appropriate for optimum results. In accordance with the invention, a nucleic acid may exhibit at least from about 80% to about 100% sequence identity with one or more nucleic acid molecules as described herein, for example at least from about 80%, about 85%, about 90%, about 95%, about 98%, about 99%, or about 100% sequence identity. One of skill in the art will understand that stringency may be altered as appropriate to ensure optimum results.

As used herein, the terms “sequence identity,” “sequence similarity,” or “homology” are used to describe sequence relationships between two or more nucleotide sequences. The percentage of “sequence identity” between two sequences is determined by comparing two optimally aligned sequences over a specific number of nucleotides, wherein the portion of the sequence in the comparison window may comprise additions or deletions (i.e., gaps) as compared to a reference sequence. Two sequences are said to be identical if nucleotides at every position are the same. A nucleotide sequence when observed in the 5′ to 3′ direction is said to be a “complement” of, or complementary to, a second nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence.

SEQ ID NOS: 1, 2, 5, 6, 8, 9, 10, 11, 12, or 13 set forth particular primer pairs. Specifically disclosed are variants of these primers which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Stated another way, disclosed are primers which differ by 1, 2, 3, 4, 5, 6, 7, 8, or 10 nucleotides from SEQ ID NOS: 1, 2, 5, 6, 8, 9, 10, 11, 12, or 13. Those of skill in the art readily understand how to determine variations that can occur in the primers which still allow for the retention of their functionality.

In some embodiments, the probe of any preceding aspect or any oligonucleotide of the present disclosure may be detectably labeled. Detectable labels include, but are not limited to, radiolabels, fluorochromes, including fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxy fluorescein (6-FAM) or N,N,N′,N′-tetramethyl-6-carboxyrho-damine (TAMRA); radioactive labels such as 32P, 35S, and 3H), and the like. In some embodiments, the detectable label is a 5′ label, 3′ label, or an internal label. In some embodiments, the probes of any preceding aspect further comprises a quencher label. As used herein, a “quencher” refers to a molecule that reduces the fluorescence intensity of a fluorophore (a molecule that emits light upon excitation) by interacting with it. The interaction between fluorophore and quencher involves an energy transfer or other mechanisms that cause the fluorophore to return to its ground state without emitting light. In some embodiments, the quencher is a 5′ quencher, a 3′ quencher, or an internal quencher. Non-limiting examples of quenchers include TAMRA, Dabcyl, Black Hole Quenchers (BHQ, including but not limited to 3IAbRQSp and 3IABkFQ), and the minor groove binder (MGB). In some embodiments, a detectable label may involve multiple steps (e.g., biotin-avidin, hapten-anti-hapten antibody, and the like). A primer useful in accordance with the invention may be identical to a particular bacterial target nucleic acid sequence and different from other bacterial sequences. In another embodiment, a primer and/or probe useful in accordance with the invention may enable distinction between a nucleic acid sequence from a virulent STEC strain, such as in O157, O26, O45, O103, O111, O121, O145, O22, O55, O64, O86, and O147 (but not limited to these serogroups). In some embodiments, the kit detects the one or more virulent genes directly from a single E. coli cell.

The invention further provides diagnostic reagents and kits comprising one or more such reagents or components for use in a variety of diagnostic assays, including for example, nucleic acid assays, e.g., PCR or RT-PCR assays. Such kits may preferably include at least a first primer pair of any preceding aspect, and means for detecting or visualizing amplification of a target sequence. In some embodiments, such a kit may contain multiple primer pairs as described herein for the purpose of performing multiplex PCR or RT-PCR for detection of multiple target sequences. Primer pairs may be provided in lyophilized, desiccated, or dried form, or may be provided in an aqueous solution or other liquid media appropriate for use in accordance with the invention.

Kits may also include additional reagents, e.g., PCR components, such as salts including MgCl₂, a polymerase enzyme, deoxyribonucleotides, and the like, reagents for DNA isolation, or enrichment of a biological sample, including for example media such as water, saline, BHI, TSB, mTSB, BPW, or the like, as described herein. Such reagents or components are well known in the art. Where appropriate, reagents included with such a kit may be provided either in the same container or media as the primer pair or multiple primer pairs or may alternatively be placed in a second or additional distinct container into which the additional composition or reagents may be placed and suitably aliquoted. Alternatively, reagents may be provided in a single container means.

It should be noted that the present disclosure provides advantages and improvements over existing methods in the art. The kit, methods, primers, and probes of the present disclosure provide a significant contribution to the beef industry (i.e., Tyson, Cargill, JBS) enabling beef samples to be screened within one day and released into commerce. Regarding existing testing methods, false-positive test results using the existing method make the beef industry lose 41 million dollars each year. The methods and kits of the present disclosure provide means for preventing such losses to the beef industry. Further, the methods and assays of the present disclosure help the food industry screen and avoid other virulent E. coli strains currently not on the federal radar.

In some embodiments, the modified nucleic acid comprises a chemically modified nucleobase. In some embodiments, the chemically modified nucleobase is selected from peptide nucleic acid (PNA) base. In some embodiments, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N¹-methylpseudouridine (me¹Ψ), N⁶-methyladenosine (me⁶A), or thienoguanosine (^{th}G). In some embodiments, the chemically modified nucleobase is selected from 5-methoxyuridine (5moU), pseudouridine (Ψ), and N¹-methylpseudouridine (me¹Ψ). In some embodiments, the chemically modified nucleobase is 5-methoxyuridine (5moU). In some embodiments, the chemically modified nucleobase is pseudouridine (Ψ). In some embodiments, the chemically modified nucleobase is N¹-methylpseudouridine (me¹Ψ). In some embodiments, the at least one chemically modified nucleobase comprises N¹-methylpseudouridine (me¹Ψ) and 5-methylcytidine (5meC). In some embodiments, the at least one chemically modified nucleobase comprises pseudouridine (Ψ) and 5-methylcytidine (5meC). In some embodiments, the at least one chemically modified nucleobase comprises 5-methyluridine (5-meU) and 5-methoxycytidine (5moC). In some embodiments, the at least one chemically modified nucleobase comprises 5-methyluridine (5-meU) and 5-hydroxymethylcytidine (5hmC).

In some embodiments, the modified nucleic acid comprises a chemically modified ribose. In some embodiments, the chemically modified ribose is selected from 2′-O-methyl (2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl (2′-O-ME), 2′-O-Allyl, 2′-O-Ethylamine, 2′-O-Cyanoethyl, Locked nucleic acid (LNA), Unlocked nucleic acid (UNA), Methylene-cLNA, N-MeO-amino BNA, or N-MeO-aminooxy BNA. In some embodiments, the chemically modified ribose is selected from 2′-O-methyl (2′-O-Me) or 2′-Fluoro (2′-F).

In some embodiments, the modified nucleic acid comprises a chemically modified phosphodiester linkage. In some embodiments, the chemically modified phosphodiester linkage is selected from Phosphorothioate (PS), Boranophosphate, phosphodithioate (PS2), 3′,5′-amide, N3′-phosphoramidate (NP), Phosphodiester (PO), or 2′,5′-phosphodiester (2′,5′-PO). In some embodiments, the chemically modified phosphodiester linkage is phosphorothioate.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Digital PCR Assay

A Qiagen digital PCR was used to enable the performance sample testing without any DNA isolation, and generates virulence gene data from a single cell. The standardized protocol entails the enrichment of beef samples for 8 hours (other methods use 10-15 hours), taking 1-3 mL of enrichment, followed by centrifugation to harvest bacterial cells. These cells are dissolved in a diluent, and 5-10 μl of bacterial sample is directly used for the digital PCR assay. The assay divides bacterial cells into individual partitions and performs PCR in each partition, enabling the detection of virulence gene signals from individual cells.

An EHEC testing workflow was standardized that simplifies testing protocol, eliminates the DNA extraction step, shortens enrichment time by 4-6 hours, and, most importantly, enables the detection of virulence genes (eae, stx, and stx subtypes) in a target bacterial cell.

This is a major improvement compared to the commonly used method by the FSIS or beef industry, which entails a 10-15 hours enrichment period, DNA extraction, and real-time PCR, which detects the presence of the eae gene from other Gram-negative bacteria and stx gene from bacteriophage resulting in a high (81-100%) percentage of false-positive results.

### General Utility

The beef industry and USDA-FSIS are the two major groups that benefit from this method.

### Technical Description

Shiga toxin-producing Escherichia coli (STEC) are foodborne pathogens of great concern. In the United States, E. coli O157:H7 and six non-O157 STEC serogroups (i.e., O26, O45, O103, O111, O121, and O145) are considered adulterants in non-intact beef.

The virulent strains of STEC serogroups possess the Shiga toxin gene (stx) and the adherence factor intimin (eae) gene. These two genes are considered essential for causing severe human infection. Human infection by a virulent STEC strain may result in acute illness, bloody diarrhea, hemorrhagic uremic syndrome, and renal failure. E. coli strains lacking these genes can be considered avirulent. These avirulent strains are not a concern for the regulatory agency or the industry. However, they are responsible for false positive test results by current testing methods.

According to a USDA, FSIS Office of Public Health Science 2018 report, the false positive rates of potential screening were 93% for beef manufacturing trimming, 81% for ground beef, 100% for bench trim, and 94% for other components. This high false-positive rate results in an annual loss of approximately $47 million for the beef industry.

Currently, there are two PCR technologies for performing absolute quantification, i.e., droplet digital PCR (ddPCR) and partition-based digital PCR (dPCR). The droplet digital PCR (ddPCR) (Bio-Rad Laboratories, California) is a commercially available technology that partitions the PCR reaction mixture in the form of picoliter droplets produced via water-oil emulsion. A major limitation of the droplet digital PCR method is that the whole process requires multiple supporting instruments, which include a droplet generator cartridge holder, droplet generator (i.e., DG8 or DG32), thermal cycler (i.e., T200), droplet reader, and QuantaSoft for data acquisition and analysis software.

This drastically increases the total cost of the instrument when compared to the cost of a real-time PCR or partition-based digital PCR, making it out of reach for many food testing laboratories. Further, the droplet digital PCR can take close to five hours to complete a run. Thus, making it expensive and a slower technology.

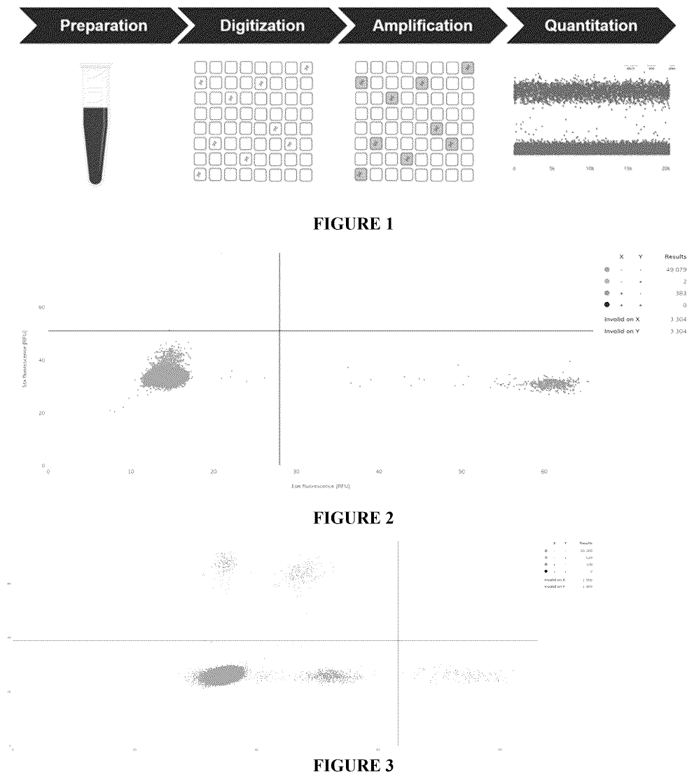

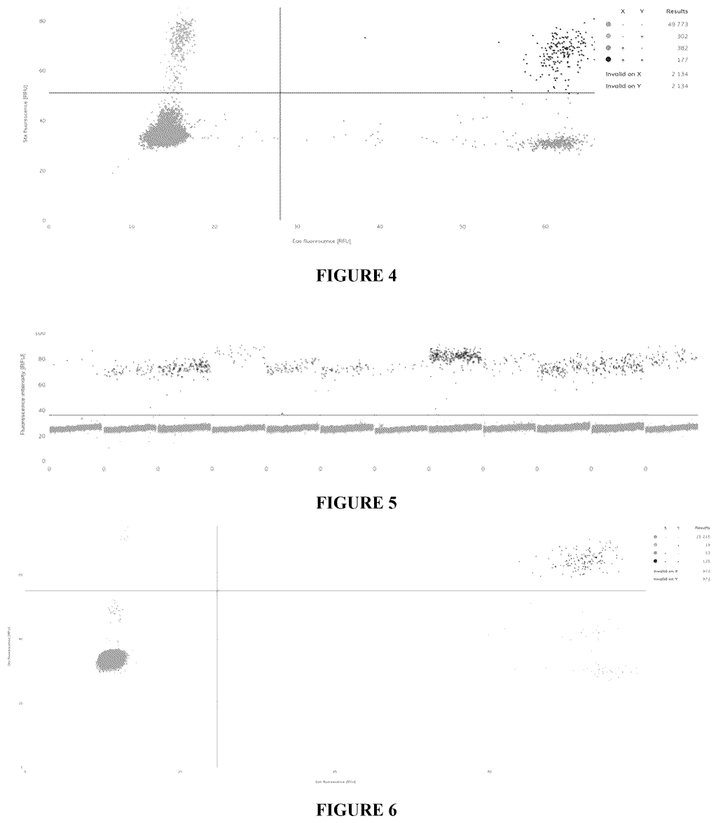

Over the years, the technology for partition-based digital PCR (dPCR) has improved, resulting in smaller, integrated, and affordable instruments that are robust, generate absolute quantification data, and have a similar instrument price point compared to a real-time PCR. Facilitating, the development and application of digital PCR methods for the detection and absolute quantification of pathogens. The partition-based dPCR assay works by partitioning a DNA sample with a reaction mix (master mix, primer, probe, DNA, and water) on a nanoplate. The number of partitions per sample depends on the nanoplate selection (i.e., 8,500 or 26,000 partitions plate). Subsequently, after the partitioning of the reaction on a nanoplate, the connecting channels between the partitions are sealed by a pressure-controlled rolling process, locking a nanoliter reaction volume in each partition, which may or may not have the target DNA molecule. After sealing, thermocycling is performed to amplify specific targets. At the end of the thermocycling, the plates are imaged to measure the fluorescence data from each partition, which is used to identify the number of positive and negative partitions. The positive and negative partition data points are used to calculate the target genome copies/μl of the DNA sample (FIG. 1).

Method: A simple digital PCR assay was standardized that can help the beef industry rapidly screen beef samples for the presence of bacterial cells that are double-positive for stx and eae genes. This step is crucial for the beef industry as they must perform rapid testing before the product is shipped into commerce. The longer the product sits in the freezer, the more it will lose its value.

Version 1 of the workflow entails inoculation of 325 g ground beef samples with a virulent or avirulent, or a mixture of two avirulent E. coli strains at 10 CFU/325 g. The beef sample, after inoculation, was stored in a refrigerator at 4° C. for 24 hours. Samples after storage were enriched with 1 L of mTSB broth at 42° C. for 6 hours. Three mL of enrichment were collected at the end of the enrichment period. The bacterial cells were harvested from the enrichment by centrifugation. The supernatant was discarded, and the cell pellet was dissolved by vortexing in 100-1000 μl of diluent. 5-10 μl of bacterial cells were used for the digital PCR reaction. The digital PCR reaction was performed using the stx and eae primers and probes listed in the MLG 5C.03, and a new primer probe for the detection of stx 2C was added to the reaction.

The amplification condition for the assay included initial denaturation at 95 C° for 10 minutes, followed by the 40 cycles at 95 C° for 10 seconds and 59° C. for 1 min. The plate was imaged in the yellow, red, and crimson channels. 1-D and 2-D scatter plots were used for the data analysis.

The results from the 2D scatter plot clearly demonstrate the assay's ability to differentiate samples inoculated with mixed samples.

### Example 2: Direct Digital PCR Assay for Screening of Shiga Toxin-Producing Escherichia coli

Shiga toxin-producing Escherichia coli (STEC) strains are characterized by the presence of Shiga toxin and intimin genes. They have a low infective dose of 1 to 10 cells, and their ingestion causes from mild gastrointestinal discomfort to severe manifestations, including bloody diarrhea and kidney failure. Currently, the United States Department of Agriculture (USDA) has a zero-tolerance policy for the seven STEC (Top7 STEC) serogroups in beef (i.e., O157, O26, O45, O103, O111, O121, and O145). Therefore, beef samples must be tested before their release in commerce.

The USDA-Food Safety Inspection Services (FSIS) MLG 5C.03 is the reference method for detecting the Top7 STEC in meat products and uses four multiplex polymerase chain reaction (PCR) reactions. This method relies on a sequential approach, starting with the first screening for stx and eae genes in beef enrichments. Samples testing positive are further tested by two multiplex PCR assays for the identification of contaminating STEC serogroups. Enrichments that are stx⁺, eae⁺, and O-group⁺ are processed using the culture-based method to isolate contaminating strains. If a pure culture isolate is obtained, DNA from the isolate is tested for the presence of stx and eae genes. This process, from beef enrichment to final culture confirmation, can take up to 5 days.

The limitations of these standard testing methods are that the virulence genes used for STEC screening are not specific to STEC strains and can be found in other non-pathogenic bacteria. The presence of these virulence genes in other genera interferes with STEC detection assays and is responsible for a high number of false positives, incurring large financial loss to the beef processors.

The second limitation of the STEC testing method is its inability to differentiate between the pathogenic (disease-causing) and non-pathogenic strains of Top7 STEC serogroup strains. The non-pathogenic strains have lost one or both virulence genes, and the FSIS zero-tolerance policy does not apply to these strains.

These two factors are responsible for high false-positive screen test results, which has been recently acknowledged by the USDA, further emphasizing a need for a new STEC testing method that looks for virulence genes in a single bacterial cell.

Even when a product tests false-positive (i.e., has no pathogen), the product cannot be released in commerce and is sold at a very low price. It costs a processor $30,000 in losses on that specific product lot, and they also incur additional financial losses on the two shoulder lots as they cannot be completely sure about the two shoulder lots. Annually, these losses cost the beef industry more than $41 million.

Meat industry stakeholders were recently interviewed to identify their research needs and pain points. Large beef processors like Tyson Foods and Cargill generate around 40-60 samples/hour for testing and internally test around 700 samples daily. In contrast, small processors (Florida Beef Inc.) ship around 100 samples daily to independent testing laboratories for STEC testing. Due to the sheer volume of samples and short shelf-life of beef products, all beef testing labs (i.e., regulatory, processing facility, and independent testing labs) need a method that is robust, simple, very high-throughput method (parallelly capable of testing 100s of beef samples), with the least amount of hands-on sample preparation steps (e.g., run tests without DNA isolation step, as it can take from 30-60 min to isolates DNA from 48 beef enrichment samples) and can preferably generate results within 8-hour workdays.

Thus, the present disclosure describes assays and methods developed in response to needs expressed by beef industry stakeholders, processors, beef testing laboratories, and USDA Agricultural Research Services (USDA-ARS) beef safety laboratories.

Therefore, the present disclosure presents a simple, high-throughput STEC testing method that can test for the presence of E. coli virulence genes (stx, eae, O157:H7) within one cell and eliminate any false-positive signal generated due to the presence of bacteriophage and non-pathogenic Enterobacteriaceae DNA present in beef enrichments. The present disclosure aims to: (1) standardize a direct digital PCR assay to detect pathogenic STEC strains; and (2) validate the direct digital PCR assay using laboratory-inoculated beef samples.

The assay developed herein was able to detect E. coli O157:H7 and a broad range of STEC strains following an eight-hour enrichment period. The assay also worked directly on bacterial cells harvested from enrichment. Further, this assay simplified the STEC testing procedure and reduced false-positive results. These advancements in STEC detection are significant for beef industry stakeholders and have an impact on food safety.

Under the United States Federal Meat Inspection Act (FMIA), codified at 21 U.S.C. 601(m)(1), specific regulations are established regarding the safety and quality of meat products in the United States. According to these regulations, all raw non-intact beef, as well as raw intact beef intended for processing into raw non-intact products, are deemed adulterated and thus prohibited from being released into commerce if they are found to be contaminated with any of the seven specific serogroups of E. coli. These serogroups include E. coli O157, O26, O45, O103, O111, O121, and O145.

The significance of this regulation lies in the pathogenic nature of these E. coli serogroups, particularly those that possess both the Shiga toxin (stx) and intimin (eae) genes. The presence of these genes in the same bacterial cell enhances the potential and severity of infection. Shiga toxin can cause severe gastrointestinal distress and complications, while intimin is associated with the ability of the bacteria to adhere to and invade intestinal cells, further contributing to the virulence of these pathogens.

As a result, any beef products found to be contaminated with these specific E. coli strains cannot be sold or distributed in the marketplace, emphasizing the importance of Shiga toxin-producing Escherichia coli (STEC) testing for the beef industry. This regulation aims to protect public health by ensuring that contaminated meat products do not enter the food supply, thereby minimizing the risk of foodborne illness outbreaks associated with these dangerous pathogens.

Shiga toxin-producing Escherichia coli strains are notable pathogens characterized by the presence of Shiga toxin, which is encoded by the stx gene. This potent toxin has the ability to cleave red blood cells, leading to severe health consequences. Additionally, STEC strains produce intimin, a protein encoded by the eae gene, which facilitates robust attachment of the bacteria to host cells, enhancing their ability to establish infection.

After ingestion, STEC strains survive and pass through the acidic environment of the stomach and travel to the small intestine, where they begin to establish an infection. The infection process starts with the eae gene expressing a protein called “intimin”. The intimin protein plays an important role in the bacteria's ability to strongly adhere to the host epithelial cells of the intestinal lining. The interaction of the intimin protein with the host cells is mediated by the “Tir” receptor, which is injected into the host cell via a type III secretion system.

Once E. coli has attached to the host's intestinal epithelial cells, it rearranges the cell's actin cytoskeleton, causing the formation of pedestals. These pedestals are cytoskeletal protrusions that essentially “lift” the bacteria off the surface of the intestinal cell, creating a more stable attachment site and enhancing the bacterium's ability to resist flushing by intestinal motility. This process also damages the epithelial cells, disrupting the normal intestinal barrier, which can facilitate the bacteria's access to deeper tissues and the bloodstream.

Once colonization is established, STEC bacteria produce Shiga toxin (encoded by the stx gene). Shiga toxin is a potent cytotoxin that can devastate the local intestinal cells and distant organs (i.e., kidney). The toxin binds to globotriaosylceramide receptors on the surface of the intestinal lining and kidney. This binding allows the toxin to be internalized by the host cell, where it prevents protein production, leading to cell death through apoptosis or necrosis.

One of the alarming features of STEC is their low infectious dose; as few as 1 to 10 bacterial cells can cause illness in humans. Upon ingestion, these strains can result in a spectrum of symptoms that range from mild gastrointestinal discomfort to severe manifestations, including bloody diarrhea and kidney failure. Individuals may experience hemorrhagic colitis (HC), a painful inflammation of the colon, which can progress to hemolytic-uremic syndrome (HUS) a life-threatening condition that can result in kidney failure and in extreme cases loss of life.

In the United States, STEC strains are responsible for many foodborne outbreaks, affecting a wide array of food products. STEC outbreaks have been associated with various red meat products, unpasteurized raw milk products, contaminated fresh produce, grains, and flour, highlighting the diverse sources of food in which they can be present. According to estimates, STEC causes approximately 2.8 million infections globally each year, with around 265,000 of those infections occurring within the U.S.

To combat this serious public health threat posed by STEC, the United States Department of Agriculture (USDA) enforces a strict zero-tolerance policy for the seven specific STEC serogroups, often referred to as the “Top7 STEC”, in beef products. These serogroups include O157, O26, O45, O103, O111, O121, and O145. Consequently, beef samples must undergo rigorous testing for these pathogens before they can be released into the marketplace, underscoring the importance of food safety regulations in protecting public health and preventing foodborne illness outbreaks.

Beyond the well-known Top7 STEC serogroups, which include O157, O26, O45, O103, O111, O121, and O145, there exists a diverse array of other E. coli serogroups that pose significant health risks. The Top7 are prioritized primarily due to their historical association, higher frequency of isolation from beef samples, their association with severe outbreaks, and high morbidity rates. However, emerging E. coli serogroups such as O22, O55, O64, O86, and O147 have also been reported to cause human illness and have been isolated from foodborne outbreaks, and there is a conversation brewing up to declare them as an adulterant in beef samples. These E. coli serogroups beyond the Top7 are broadly referred to as “non-Top7 STEC”. This broadening spectrum of pathogenic E. coli serogroups with equal severity of infection necessitates a need for a single STEC testing assay that can detect Top7 and also the non-Top7 STEC strains.

The identification of non-Top7 STEC contamination in food is crucial for safeguarding public health, as these strains can also lead to infection of equal severity, including HC and HUS, and it can result in acute kidney failure and other life-threatening complications, especially in vulnerable populations such as children and the elderly. Therefore, detection of these non-Top7 STEC contamination in food samples is essential for comprehensive surveillance and effective outbreak management.

Currently, non-Top7 serogroups can be present in food products or environmental reservoirs, and these products and samples are not subjected to any federal agency regulations. Due to a lack of effective diagnostic tests, these serogroups are slipping through federal and private testing laboratories and are causing foodborne outbreaks and illnesses. Therefore, enhancing federal agencies, and other food testing laboratories' ability to identify these emerging serogroups is imperative.

Current methods for the identification of these non-Top7 STEC strains commonly employ a combination of serotyping techniques alongside whole-genome sequencing methods, which are complex and not suited for large-scale testing operations. Food testing labs need testing technologies that can enable rapid identification and characterization of a broader range of STEC strains, facilitating timely removal of contaminated food products from commerce.

Strengthening STEC detection methods and expanding surveillance to additionally include non-Top7 STEC serogroups in a single assay plays a key role in preventing outbreaks. By staying ahead of emerging threats and enhancing the understanding of these pathogens, a safeguard to public health can maintain the brand image of the United States beef.

### Standard Testing Methods

The USDA-Food Safety Inspection Service (FSIS) Microbiology Laboratory Guidebook (MLG) 5C.03 serves as the official reference method for the detection of the Top7 STEC in meat products. This method employs four multiplex PCR reactions, which are designed to facilitate the detection of Top7 STEC-associated gene targets.

The screening and isolation method for detecting STEC begins with a crucial step. The beef samples or swabs are enriched in a modified tryptic soy broth medium. This broth is specifically formulated to create an optimal environment for the growth of STEC while slowing down the growth of other non-target bacteria. The enrichment process increases the likelihood of detecting low levels (0.5-1 CFU/325 g ground beef or 375 g beef trim) of STEC that may be present in food products or environmental samples.

### Screen Positive

Once the samples have been enriched, DNA from samples are isolated and screened with a PCR test that targets the stx and eae genes, key genetic markers that are associated with STEC strains. The detection of these genes in DNA of the test samples indicates possible presence of a pathogenic E. coli strain. These samples are called “screen positive”.

These screen-positive samples are then tested by a two or three multiplex real-time PCR assay for the serogroup identification of the contaminating STEC strain. If any of these real-time PCR assays generate a signal for a STEC serogroup, the enrichment broth is further processed by immunomagnetic separation (IMS) beads for the isolation of the TOP7 STEC. In this process, 100 μL of IMS beads is added to the 1 mL of enriched broth. The beads bind to STEC cells, allowing for their separation from the food matrix through the application of a magnetic field. This step enhances the purity, drastically reduces the background bacterial load, and concentration of the target STEC, ensuring that subsequent tests are more accurate.

The magnetic beads with STEC cells are then plated onto a chromogenic medium known as modified Rainbow Agar (mRBA). This agar is tailored to suppress other bacterial species, promote the growth of STEC, and generate specific-colored colonies, making it easier to identify and isolate the target organisms.

Samples that yield positive results when screened with real-time PCR for STEC serogroup are classified as “potential positive”. This designation shows that the sample may contain one or more of the seven target STEC serogroups, or presence of a non-pathogenic E. coli strain indicating the need for further testing and confirmation.

### Presumptive Positive

After identifying positive samples, the next step involves examining the colonies that have developed on the mRBA plates. These colonies are tested with the latex agglutination test. The antisera contain monoclonal antibodies for the detection of E. coli serogroup specific O-antigens protein, specific markers that are used to identify E. coli serogroups. In this test, a part of the isolated bacterial colony is transferred to a slide and mixed with a drop of latex beads with antibodies against the one STEC O-antigen. If agglutination occurs, it indicates a positive reaction, confirming the isolation of a specific STEC serogroup.

Simultaneously, the colonies undergo presumptive rapid screening with multiplex real-time PCR. This PCR method is employed to further confirm the presence of STEC-related genetic material, providing additional support for the initial findings from the enrichment and plating stages.

When one or more typical colonies from the mRBA plates show agglutination with STEC latex agglutination reagents and test positive by the PCR assay, these samples are now classified as “presumptive positive”. This classification indicates a strong likelihood that these colonies are of pathogenic STEC strains.

To confirm these findings, agglutination-positive colonies are taken with a sterile loop and then streaked onto a trypticase soy agar supplemented with 5% sheep blood (SBA). This enriched medium supports the growth of a wider variety of bacteria and allows for the observation of hemolytic activity, which is a characteristic feature of most STEC strains.

### Confirmed Positive

The colonies growing on the SBA plates are subjected to additional testing for the presence of O-antigens using a further agglutination test. This step ensures that the colonies identified as presumptive positive are indeed associated with the target serogroups. After confirming the presence of O-antigens, the colonies are identified using the Bruker® MALDI Biotyper (MBT), an advanced mass spectrometry system that provides precise identification of bacterial species based on their unique protein profiles.

Additionally, confirmation rapid screening PCR is performed to validate the presence of STEC serogroup-specific genes, further ensuring the accuracy of the identification process.

A sample that confirms as an E. coli isolate containing both the stx gene and the eae gene, along with one of the specified target serogroups, is classified as CONFIRMED STEC positive. This designation signifies a definitive identification of a pathogenic STEC strain, indicating its potential to cause severe illness.

Availability of an accurate and simple pathogen detection assay is crucial for implementing an effective federal meat inspection program. Hence, accurate STEC testing methods are needed for protecting human health from a wide range of STEC strains. These assays enable health authorities to be better equipped to implement food safety programs and effectively eliminate contaminated samples from processing plants and commerce.

### STEC Testing Programs and Associated Challenges

In 2023, in response to a troubling rise in cases of Shiga toxin-producing Escherichia coli (STEC), the USDA-FSIS expanded its verification testing program. Initially focused solely on the detection of E. coli O157:H7, the program was broadened to include six additional STEC serogroups: O26, O45, O103, O111, O121, and O145. This expansion now includes raw ground beef, bench trim, and other components of raw ground beef collected at official meat processing establishments.

Despite this initiative, many in the beef industry remain hesitant to adopt the expanded testing protocols. Currently, processors predominantly test for E. coli O157:H7, largely neglecting the additional six serogroups. Industry representatives argue that the testing for these pathogens is prohibitively expensive, and they generate 80-100% false-positive results. Leading beef industry stakeholders have expressed their frustration with STEC testing methods and mentioned “Current assays are not good enough, and we don't want to spend good money for false-positive results.” This reluctance to adopt broader testing practices underscores a significant challenge in enhancing food safety within the beef industry.

The recent expansion of the USDA-FSIS verification testing program marks a crucial step in addressing the rising cases of STEC. The initiative now includes the Top7 STEC serogroups, which, although is a significant change, but it does not encompass the entire spectrum of STEC that can pose health risks.

While the Top7 serogroups are the most recognized due to their association with severe outbreaks, numerous other STEC serogroups exist that can also lead to severe infections and complications similar to those caused by the Top7. Some of these emerging serogroups such as O22, O55, O64, O86, and O147, have been implicated in human illnesses and outbreaks, indicating that the severe disease extends beyond the currently prioritized serogroups.

The hesitancy of the beef industry to adopt broader testing practices emphasizes a significant gap in food safety. Although the FSIS program has expanded to include six non-O157 serogroups; as a result, many potentially harmful serogroups are currently passing unnoticed through the testing lab, leading to a false sense of food safety.

Infections caused by non-O157 STEC strains are equally severe as those caused by E. coli O157:H7, which highlights the need for a single assay that covers all Top7 strains and also goes beyond these serogroups.

The industry's current focus on E. coli O157:H7 is understandable, given its historical prevalence and severity. However, this narrow approach can hinder the detection of non-Top7 serogroups that are just as pathogenic. The argument that current testing methods result in high false-positive rates further complicates the issue, but it underscores the need for improved assay development and validation to ensure accurate detection of a broader range of STEC.

### Economic Impact of False-Positive Results

The financial ramifications of false-positive test results are substantial. When a product tests false-positive for STEC but is ultimately found to contain no pathogenic bacteria, it still cannot be released into the marketplace. Instead, such products often have to be sold at significantly reduced prices, leading to substantial losses. On average, a processor may incur losses of approximately $30,000 on the specific product lot that was tested positive. Moreover, these losses extend to two additional shoulder lots that may also be affected, compounding the economic impact. Collectively, these factors contribute to an estimated annual loss exceeding $41 million for the beef industry as a whole.

To effectively mitigate the occurrence of false-positive STEC test results, enhance overall food safety, reduce the burden of STEC infections, and encourage the adoption of testing within the industry, there is a pressing need for the development of a reliable and accurate method for STEC detection. Establishing such a method would not only facilitate more extensive testing but also support the expansion and implementation of federal testing programs. Additionally, it would play a critical role in preventing outbreaks and yield significant economic benefits for industry stakeholders, aligning with public health goals such as the Healthy People 2020 initiative, which aims to achieve a target of 0.6 STEC cases per 100,000 population. Overall, addressing these challenges is essential for safeguarding public health and promoting the sustainability of the beef industry.

### STEC Beef Industry Testing Lab Needs

Recently, the inventor of the present disclosure interviewed stakeholders in the meat industry to gain insight into their research needs and identify the pain points related to food safety, specifically concerning STEC testing. The findings from the interviews revealed significant disparities between large and small beef processors regarding their testing practices, capabilities and needs.

Large processors, such as Tyson Foods and Cargill, typically conduct internal testing on approximately 700 samples each day to ensure the safety of their products. This high volume reflects their need for rapid and reliable testing methods with a short turnaround time. Non-intact beef has a shorter shelf-life, and the product must be received at the retailer's distribution facility within a few days after the grinding or trimming. On the other hand, smaller processors, like Florida Beef Inc., send around 100 samples daily to independent testing laboratories for STEC testing. Independent food testing labs (e.g., Eurofins) test around 2000 beef samples every day. This difference in sample volume highlights the varying capacities and operational challenges faced by processors and testing labs of different sizes.

Given the substantial number of samples processed daily and the perishable nature of beef, all laboratories involved (e.g., regulatory bodies, processing facilities, or independent testing labs) require a testing method that is robust, user-friendly, and capable of high throughput. Specifically, there is a pressing need for a method that allows for the parallel testing of hundreds of beef samples with minimal hands-on preparation steps. For instance, current methods that involve DNA isolation can take 30-60 min just to process 48 beef enrichment samples, which is impractical given the time constraints.

Furthermore, stakeholders expressed a strong preference for a testing approach that can generate results within a typical 8-hour workday. This requirement underscores the urgency of having timely and actionable data to ensure product safety and compliance with regulatory standards.

In response to these beef industry STEC testing needs, the present disclosure has developed STEC testing methodologies best suited for large scale testing laboratories. The initiative herein is informed by the insights gathered from processors, beef testing laboratories, and USDA Agricultural Research Services (USDA-ARS) beef safety laboratories. The inventors, who has a history of collaboration with these entities, will continue to engage with them to ensure the project aligns with industry expectations and delivers practical solutions to current challenges. This collaborative effort aims to address the critical needs of the beef industry, ultimately improving beef safety and operational efficiency.

The goal of this project is to develop a simple, high-throughput testing method for testing STEC in beef samples that accurately detects the presence of virulence genes; specifically, the stx and eae genes inside a single-cell.

By developing a method that can effectively distinguish between pathogenic and non-pathogenic strains, a reliable tool is provided herein that meets the rigorous demands of the beef industry. The testing method not only facilitates the rapid identification of a broad range of pathogenic E. coli strains but also enhance overall food safety by reducing the risk of false positives. Ultimately, this project seeks to contribute to be a more efficient and accurate testing framework, ensuring that beef products are safe for consumption while supporting the operational needs of processors and laboratories alike.

### Background

The partition-based direct digital PCR for STEC detection developed in the Florida State University, Food Microbiology Laboratory, is a major breakthrough for detecting STEC in food samples. This innovative assay is designed to work directly with bacterial cells, effectively eliminating the need for traditional DNA isolation steps. By doing so, it enables the detection of pathogenic genes in a single cell. Therefore, the approach is also known as “Single Cell PCR”. The approach provides a more precise approach for the detection of STEC.

During the direct digital PCR process, the test samples, which consist of bacterial cells, are mixed with a PCR reaction mixture and are loaded on to the digital PCR nanoplate. These nanoplates are available in either 8,500 or 26,000 individual partitions. After the samples are partitioned onto the nanoplate, a pressure-controlled rolling process is employed to seal the connecting channels between the partitions. This process locks in a nanoliter reaction volume, each containing a bacterial cell or, in some cases, no cell at all.

The initial step of the PCR thermocycling involves denaturation at 95° C. for 10 min. This step is critical as it leads to the lysis of the bacterial cells, releasing their genomic DNA into the reaction mix and activation of Taq DNA polymerase. Following this, multiplex PCR amplification is performed within each individual partition using specific fluorescent probes designed to target the virulence genes (i.e., stx and eae) of interest. If a partition contains a bacterial cell that tests positive for a specific virulence gene, it generates a corresponding fluorescent signal. For instance, if a bacterial strain is positive for two virulence genes (i.e., stx and eae), the associated partition will display corresponding signals for i.e., FAM and HEX fluorescent dyes.

In the final stage of the assay, the nanoplate is imaged to capture the fluorescent signals emitted from each partition. This imaging process provides data that allows for the identification of positive and negative partitions based on the presence of fluorescence. The initial data obtained is utilized to calculate absolute quantification of the target genes. Furthermore, a detailed analysis is conducted to assess double-positive partitions through a two-dimensional (2D) analysis, which enables characterization of the virulence profile of the bacterial cell.

Overall, the Partition-based digital PCR assay represents a cutting-edge method for the rapid and accurate detection of STEC in food products. By focusing on bacterial cells directly and allowing for precise quantification of virulence genes, this approach holds great promise for improving food safety and public health by facilitating early detection and intervention in the case of contamination.

### Advantages of Direct Partition-Based Digital PCR Assay

Unlike the droplet digital PCR technology, which requires four different instruments, the partition-based direct digital PCR assay is integrated into a single instrument known as the QIAcuity One (Qiagen). This streamlined technology enables PCR reactions to be conducted directly from beef enrichment samples without the need for prior DNA isolation. This significant advancement not only saves valuable hours in sample preparation but also reduces both the cost, time and labor associated with conducting the assay.

Key advantages of this technology are its remarkable resistance to PCR inhibitors, which are common in complex food matrices (e.g., beef). This feature enhances the reliability and accuracy of the test results, minimizing the chances of false negatives that can arise from the presence of inhibitors and eliminates the need for a high-purity DNA isolation kit (i.e., DNeasy Blood and Tissue Kit, DNeasy Power Food Microbial Kit) which takes 3-4 hours to generate high-purity DNA samples. Moreover, the QIAcuity One supports multiplexing capabilities, allowing for the simultaneous detection of up to five gene targets in a single assay. This multiplexing feature greatly increases efficiency, as multiple targets can be analyzed concurrently, further streamlining the testing process.

The overall run time for the assay is approximately two and a half hours, making it a rapid solution for food safety testing. Importantly, the cost of reagent and instrument for the digital PCR assay is comparable to that of traditional real-time PCR assays, which are widely utilized in food testing. This cost-effectiveness ensures that laboratories can adopt the digital PCR approach without significant financial barriers.

The instrument is designed for high-throughput applications, the digital PCR instrument can accommodate the simultaneous operation of up to eight 96-well plates in its high-end models. This capability is particularly beneficial for laboratories processing large volumes of beef samples (i.e., 700-2000 samples a day), as it allows for efficient scaling of testing operations while maintaining accuracy and speed.

In summary, the integration of the partition-based digital PCR into the STEC detection workflow enhances both the efficiency and reliability of PCR testing in food safety. By eliminating the need for DNA isolation, reducing preparation times, and enabling multiplexing, this advanced technology is poised to significantly improve food testing processes and outcomes.

To validate the proof-of-concept assay, a series of experiments were conducted using ground beef samples that had initially screened negative for the presence of STEC according to the standard FSIS MLG 5C.03 method. For this study, the samples were inoculated with 5-10 colony-forming units (CFU) per 20 grams of ground beef spread on a micro tally swab.

Following inoculation, the samples were subjected to cold stress by being stored at 4° C. for 24 hours. This step is critical as it mimics conditions that may occur during storage and transportation, affecting the lag-phase of the inoculated bacteria. After the cold-stress period, the samples underwent aerobic enrichment by being incubated in 200 mL of modified tryptic soy broth (mTSB) (Hardy Diagnostics) at 35° C. for 8 hours. This enrichment step is designed to promote the growth of the inoculated bacteria, enhancing their detectability in subsequent analyses.

At the end of the incubation period, the enrichment broth was collected for further processing. The samples were then centrifuged to harvest the bacterial cells effectively. After centrifugation, the supernatant was discarded, and the bacterial cells were vortexed with a diluent to resuspend them for their analysis.

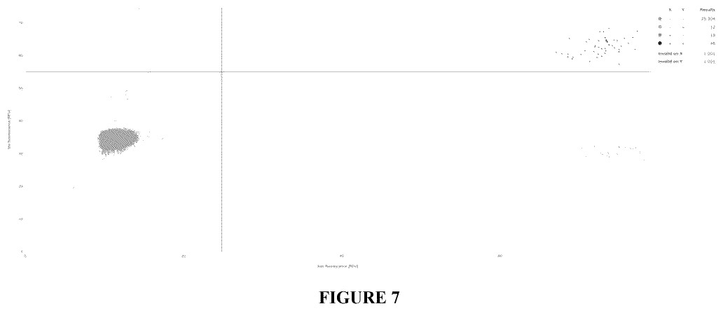

Subsequently, the direct digital PCR assay was optimized and performed using 5-10 μL of the resuspended bacterial cells. The results from the assay were promising, demonstrating its capability to accurately identify beef samples inoculated with a pathogenic E. coli strain that tested positive for both the stx and eae virulence genes (denoted as stx⁺ and eae⁺) (as illustrated in FIG. 2, 3, 4). In contrast, the assay also successfully identified a non-pathogenic E. coli strain that was positive for the eae gene but negative for the stx gene (denoted as stx⁻ and eae⁺), showing no double-positive partitions, as depicted in FIG. 7.

These findings highlight the effectiveness of the assay in differentiating pathogenic from non-pathogenic E. coli strains in food samples. This capability is crucial not only for enhancing food safety protocols but also for ensuring the accurate detection of pathogenic E. coli strains.

To standardize a direct multiplex digital PCR assay to detect pathogenic STEC strains, the following concepts were contemplated herein: (1) A direct digital PCR assay targeting the eae, stx, O157:H7 genes enables the detection of E. coli cells that are positive for crucial virulence genes and facilitate the elimination of false-positive STEC test results; and (2) A virulence gene-based approach enables a serogroup-independent screening of highly pathogenic (O157:H7) E. coli strains that are associated with severe cases.

### Bacterial Strains

FSU food microbiology culture collection boasts one of the largest collections of STEC strains available on the Florida State University campus. This comprehensive collection includes a total of 115 strains, comprising 79 non-pathogenic and 36 pathogenic STEC strains, which are distributed across 21 distinct E. coli serogroups (O-groups). The strains that were included in the standardization process were O157:H7 (n=16), O26 (n=6), O45 (n=8), O103 (n=8), O111 (n=8), O121 (n=11), and O145 (n=5) for the TOP 7. E. coli strains belonging to non-Top7 STEC serogroups were also included, O5, O15, O55, O74, O84, O98, O118, O153, O157, O172, O177, O182, and O186.

The strains in this collection are ideally suitable as they originate from beef and have been isolated from the federal red meat surveillance program. This program involves the systematic collection of beef samples from various red meat processors, followed by isolation and characterization of obtained isolates using standard MLG 5C. 03 protocol established by the USDA-FSIS.

By leveraging these well-characterized strains, the accuracy and reliability of the testing method was carefully evaluated. The diversity of the strains in terms of pathogenicity and serogroup representation provides a robust framework for standardizing as well as assay evaluation, ensuring that it can effectively detect a wide range of STEC variants. This foundation is crucial for developing a method that meets the high standards required for beef safety, and federal agencies across the world.

### DNA Extraction

Genomic DNA from overnight pure broth cultures was extracted using Extracta DNA Prep for PCR (QantaBio, MA, USA) following manufacturer instructions and the concentration of isolated DNA was measured using a NanoDrop One Spectrophotometer (Thermo Fisher, DE, USA). DNA samples were diluted to a 10 ng/μL concentration and were used for characterizing the presence of virulence genes in the strains.

### Strain Characterization Using Virulence Gene-Specific PCR

The isolates obtained from FSU food microbiology culture collection were rigorously tested for the presence of key virulence gene subtypes: eae, stx. This testing was conducted using methodologies as previously outlined by Wang et al. (2002) (Wang, G., Clark, C. G., & Rodgers, F. G. (2002). Detection in Escherichia coli of the genes encoding the major virulence factors, the genes defining the O157:H7 serotype, and components of the type 2 Shiga toxin family by multiplex PCR. Journal of Clinical Microbiology, 40(10), 3613-3619. doi.org/10.1128/JCM.40.10.3613-3619.2002) and Paton and Paton (2002) (Paton, A. W., & Paton, J. C. (2002). Direct detection and characterization of Shiga toxigenic Escherichia coli by multiplex PCR for stx1, stx2, eae, ehxA, and saa. Journal of Clinical Microbiology, 40(1), 271-274. doi.org/10.1128/JCM.40.1.271-274.2002).

### Hydrolysis Probe Design

For testing protocols, stx and eae primers and probes are available for experiments disclosed herein. These established primers have been extensively validated for the accurate detection of the two target virulence genes.

In addition, primers and hydrolysis probes for the specific detection of the E. coli O157:H7 serotype were designed using Primer3 software. This software allows for precise design of oligonucleotides tailored to the specific detection needs. To enhance the performance and annealing temperature of these probes, locked nucleic acid (LNA) bases were incorporated into the probe design.

All new probes for the detection of E. coli O157:H7 were optimized and rigorously tested using a comprehensive panel of E. coli strains, each representing different stx gene subtypes. This testing of the hydrolysis probes accurately detects only amplification for the intended O157:H7 specific gene, thereby minimizing the risk of cross-reactivity with non-target strains.

### Digital PCR Assay Standardization

A multiplex digital PCR (dPCR) assay for the simultaneous detection of the virulence genes eae, stx, O157:H7 was optimized using pure culture E. coli DNA samples. This assay standardization step is crucial for ensuring reliable and accurate detection of these key genetic markers associated with STEC.

The hydrolysis probes used for detecting each gene were labeled with distinct fluorescence dyes: FAM for eae, HEX for stx, ROX for O157:H7. This multiplex approach allowed for the concurrent detection of three gene targets in a single multiplex digital PCR reaction, enhancing the efficiency and throughput of the testing process.

The digital PCR was performed using a high-sensitivity QIAcuity 26K nanoplate (Qiagen, Hilden). Each 50 μL dPCR reaction consisted of 15 μL of 4× QIAcuity Probe PCR reagent, specific forward and reverse primers for eae, stx, O157:H7 their respective hydrolysis probes, and nuclease-free water. The reaction mixture was thoroughly mixed and loaded into the 26K nanoplate, which is designed to accommodate high-throughput applications.

Following the loading process, the nanoplate underwent priming and sealing steps. The digital PCR amplification was performed with an initial denaturation step at 95° C. for 10 min. This step activated the Taq polymerase and, in the case of directly using bacterial cells, facilitates cell lysis, thereby releasing the genomic DNA into the reaction mix locked inside a partition.

As each bacterial cell is confined to its own partition, the amplification of target genes occurs without interference from any other DNA molecules, ensuring result from a single cell and accurate quantification. After the initial denaturation, the PCR amplification consisted of 40 cycles of a two-step PCR cycle: denaturation at 95° C. for 15 seconds, followed by annealing and extension at 64° C. for 60 seconds. After the PCR amplification, fluorescent data was collected in the FAM, HEX, and ROX channels. This step is also known as “Imaging”. The data obtained was analyzed using the QIAcuity Software Suite 2.2.0.8, enabling assess to the presence and quantification of the target virulence genes effectively.

Assay Validation with Pure Culture Strains

Following the standardization of the digital PCR assay, a comprehensive assay validation was performed using a diverse set of target E. coli strains (n=115) as well as non-target Enterobacteriaceae strains (n=24). This validation process included E. coli strains that are specifically positive for O157:H7, which are critical for assessing the assay's effectiveness in detecting pathogenic strains.

In addition to the target strains, Citrobacter and Shigella were also included for assay validation protocol. These genera have been documented to interfere with existing STEC testing assays, making their inclusion essential for assessing the robustness and specificity of the method herein.

The validation process started by growing the cultures in 10 mL of tryptic soy broth (TSB) (Hardy Diagnostics) at 37° C. After an overnight incubation, these cultures were serially diluted in 9 mL of 0.1% (w/v) sterile peptone water to prepare them for testing.

For the direct digital PCR assay, 10 μL of bacterial cells were taken from the 1/1000 to 1/10,000 dilution of the overnight culture. The goal was to determine the optimal volume that results in less than 1% positive partitions on the nanoplate, ensuring a high degree of specificity in the results.

Each assay run included both a positive and a negative control (no template control, NTC). The NTC served as the baseline reference, allowing for accurate assessment of the performance of the assay.

After completing the digital PCR run, the absolute quantification data was analyzed to determine the presence or absence of the target virulence genes in the tested strains. Additionally, a 2D analysis was performed to identify double-positive cells for the stx and eae genes, further enhancing understanding of the pathogenic potential of the strains.

### Assay Comparison

To facilitate a comprehensive evaluation of the assay's performance, DNA extracted from all tested strains were simultaneously analyzed using an eae and stx duplex real-time PCR assay. This approach allowed for the concurrent detection of both virulence genes, providing a robust comparison against our digital PCR assay.

The multiplex real-time PCR assay was conducted under standardized conditions to ensure accuracy and reliability. By assessing the presence of the eae and stx genes in the same reaction, we can effectively gauge the sensitivity and specificity of our new digital PCR assay.

The data obtained from the duplex real-time PCR served as a critical benchmark for assay comparison. By analyzing the results side by side, we can evaluate the performance of the digital PCR method in terms of its ability to detect target genes accurately, as well as its capacity to minimize false-positives and negatives.

### Assay Inclusivity

Strains for inclusive testing of the assay were selected to ensure the assay's specificity. These strains were selected to represent huge E. coli serogroup and serotype diversity associated with STEC infections. Other key factors that were considered during strain selection were presence of virulence genes, association of STEC serogroups in foodborne illness outbreaks, and availability of strains from reputable culture collections.

The strains for inclusivity testing include E. coli O157:H7 (n=16), which is one of the most commonly tested STEC serotypes. Additionally, STEC strains from other STEC serogroups that are considered an adulterant in red meat were included: O26 (n=6), O45 (n=8), O103 (n=8), O111 (n=8), O121 (n=11), O145 (n=5). Further strains belonging to the non-Top 7 STEC strains spread over 13 serogroups were included to test the assay specificity.

### Assay Exclusivity

In contrast, the exclusivity panel consisted of non-target organisms closely related to E. coli, particularly those that might coexist in the beef samples or processing environments or share genetic similarities that could lead to cross-reactivity. This part of the validation confirms whether the assay can differentiate O157:H7 and STEC strains from other closely related species and avoid false-positive test results.

For the inclusivity testing phase, each strain was cultured using 10 mL TSB. The target concentration for the test strains was 100 times the LOD50 (limit of detection at which 50% of replicates are expected to test positive), which ensured the assay is evaluated under conditions that exceed the basic detection threshold. In order to achieve this, strains were diluted 10,000 times with nuclease-free water.

For exclusivity testing, each strain was grown in non-selective media (TSB), allowing maximum growth and providing a robust challenge to the assay's specificity. All strains for the specificity testing were tested in duplicate at the same dilution (10,000 times) as inclusivity testing plus one higher dilution level (1,000 times dilution).

### Performance Criteria

The sensitivity, specificity, positive predictive value (PPV), negative predictive value (NPV), test accuracy of assay was calculated as described in the FSIS Guidance for Test Kit Manufacturers. Assays showing values greater than 90% were considered suitable. All statistics and visualizations are performed using GraphPad PRISM software.

To validate the direct digital PCR assay using laboratory-inoculated beef samples, the following concepts were contemplated: (1) the assay enables the detection and differentiation of pathogenic strains of E. coli (stx⁺, eae⁺) from non-pathogenic strains of E. coli (stx⁻, eae⁺ or stx⁺, eae⁻) strains or mixture of non-pathogenic strains in beef enrichments; (2) changing the testing method from 325 g beef bags to swabs and reducing the volume of enrichment media from 1 L to 200 mL enables faster bacterial growth and standardization of a testing workflow that can be completed in an 8-hour workday; and the assay is able to detect pathogenic strains of E. coli. at the low inoculation level of 0.5 CFU/swab.

The following methods were used for the validation of the direct digital PCR assay at high and low inoculation levels:

(1) Assay Validation Using Beef Swab Samples Inoculated with Pathogenic E. coli Strains

The assay was validated by inoculating ground beef samples following USDA-FSIS guidelines for Evaluating the Performance of Pathogen Test Kit Methods. Food samples were inoculated with the pathogenic strains (stx⁺, eae⁺) of O157:H7. These strains were grown in 10 ml TSB, at 35° C. for 24 h. After this they were serially diluted enumerate strains on TSA (Hardy Diagnostics) plates, which were incubated at 35° C. for 24 h.

Ground beef samples were purchased from the local grocery store. Samples were initially tested for STEC contamination using standard USDA-FSIS MLG 5C.03 protocol. Ground beef samples testing negative were used for assay validation. Briefly, frozen beef samples were thawed inside a refrigerator. Once thawed, the MicroTally swab (FREMONTA Corp., San Jose, CA) was folded one more time while still in bag. The top was removed, and 20 grams of ground beef was weighed out and placed in the final fold of the swab and smeared onto the swab. After this was completed, the swab was inoculated with the calculated amount of inoculum. Swab samples were inoculated at high inoculation level at 5-10 CFU/swab. The inoculum volume was parallelly plated on plate count agar (PCA) (Hardy Diagnostics) plates to enumerate the inoculum levels.

After inoculation, samples were cold stressed by storing them at 4° C. for 24 hours. Samples were enriched using pre-warmed (42° C.) 200 mL of modified tryptic soya (mTSB) broth with casamino acid (10 g/L) and novobiocin (2 mg/L) at 37±1° C.

Swab samples were inoculated at high inoculation level at 5-10 CFU/swab. The inoculum volume was parallelly plated on plate count agar (PCA) (Hardy Diagnostics) plates to enumerate the inoculum levels.

The inoculation process was carefully controlled to ensure the even distribution of microorganisms throughout the sample. After inoculation, the samples were thoroughly mixed by hand kneading to promote the homogeneous distribution of the inoculum within the matrix. Before collecting enrichment samples for analysis, the bags with swabs samples were shaken to further ensure uniform dispersion. These steps were critical for reducing sampling error and to support reliable and reproducible test results.

Enrichments (2 mL) were collected from each bag at 8 hour and 15 hour time points. Enrichments samples were centrifuged to pellet the bacterial cells. Supernatant was discarded and the obtained cell pellet was diluted and used for direct digital PCR standardized as previously described.

(2) Assay Validation Using Beef Swabs Samples Inoculated with Non-Pathogenic E. coli Strains

The assay was further validated using the non-pathogenic (stx⁻, eae⁺ or stx⁺, eae⁻) E. coli strains of the Top7 STEC serogroups (i.e., O157, O26, O45, O103, O111, O121, and O145) as described in the sections above.

(3) Assay Validation Using Beef Swabs Inoculated with Mixed Culture of Non-Pathogenic E. coli Strains

To further validate the robustness of the assay, experiments were performed where each beef swab was co-inoculated with two distinct non-pathogenic E. coli strains. Specifically, one strain was negative for stx and positive for eae (strain 1: stx⁻, eae⁺), whereas other strain was positive for stx but negative for eae (strain 2: stx⁺, eae⁻). Each MicroTally swab was prepared as previously described and co-inoculated with ˜10 CFU of each strain to create a mixed culture environment. Swabs were stressed, enriched, 2 mL enrichment were collected and used for the assay validation.

Direct digital PCR assay's ability for identifying and distinguishing between pathogenic and non-pathogenic cells in mixed culture was evaluated.

By including this additional layer of validation, the aim is to strengthen the reliability of the digital PCR assay for detecting STEC. This rigorous approach ensured that the assay not only identifies pathogenic strains but also accurately distinguishes them from non-pathogenic counterparts.

### (4) Assay Comparison

The isolation of DNA from enriched food samples was isolated following established protocols with slight modifications to optimize the process for specific needs. In brief, 200 μL of the enrichment broth was transferred into a centrifuge tube, where the samples were subjected to centrifugation at 4,000×g for 5 min. This step effectively harvested the bacterial cells from the enrichment and allowed for efficient DNA extraction using Extracta DNA Prep (Quantabio) following manufacturer's instructions. Resulting DNA samples were quantified using a NanoDrop spectrophotometer (Thermo Scientific) and diluted to 10 ng/μL to facilitate consistent testing across all samples.

For comparative analysis, all DNA samples derived from the beef enrichments were tested in parallel using an eae and stx duplex real-time PCR assay.

Samples that test positive for both the stx and eae genes through real-time PCR were classified as indicative of a pathogenic STEC strain. In contrast, those enrichments that show double-positive results for the stx and eae genes in the 2D analysis of the direct digital PCR were similarly classified as signals for the presence of pathogenic strains in the beef samples.

### (5) Low Inoculation Levels

Assay evaluation at low inoculation level (0.5 CFU/swab) was performed in accordance with AOAC International guidelines to evaluate the performance of a direct digital PCR assay for the detection of low levels of STEC in beef swabs. The primary objective of this validation was to assess the assay's sensitivity and reliability for identifying E. coli O157:H7 cells at very low concentrations i.e., 0.5 CFU/swab. This level of sensitivity is critical, given that even one STEC cell in meat can cause fatal infection.

Herein, MicroTally swabs with beef were inoculated at three contamination levels: high, fractional positive, and negative. For the high contamination level, five replicates were tested to ensure that the assay consistently detects the target organism when present at elevated concentrations (10 CFU/swab). Twenty MicroTally swabs were inoculated at low inoculation level of 0.5 CFU/swab. This level of inoculation is also known as fractional-recovery level, where only 25-75% of replicates yield positive results. This level is especially important for establishing the assay's performance in a realistic beef processing environment, where samples are contaminated at very low levels. Finally, five replicates were not inoculated and acted as uninoculated control samples.

### (6) Performance Evaluation

The principal metric used to evaluate performance in this study is the Probability of Detection (POD), a standard measure in microbiological method validation. POD is defined as the proportion of test portions yielding a positive result for a qualitative method, at a given analyte concentration and within a specific matrix. As a concentration-dependent measure, POD reflects how well a method performs across a range of contamination levels. The POD value is calculated by dividing the number of positive test outcomes by the total number of replicates tested at that level. To ensure statistical rigor, POD estimates were reported with 95% confidence intervals, which provide an estimate of the reliability and precision of the detection rate. This statistical framework allows for objective comparison between different matrices, contamination levels, or methodological approaches, and is consistent with AOAC and international standards for method validation.

### (7) Assay Comparison and Statistical Analysis

The effectiveness of O157:H7 and STEC screening was assessed by comparing the results obtained from the direct digital PCR assay with those from the duplex real-time PCR assay. This comparative analysis was conducted using GraphPad Prism (GraphPad Software, La Jolla, CA).

To evaluate the differences between the two methods, a two-tailed Fisher's exact test was employed, which is particularly suited for comparing categorical data. This statistical test enables determining whether there are significant differences in the detection rates of pathogenic strains between the two assays.

All analyses were conducted using GraphPad Prism Version 10.1.2. Statistical significance at P≤0.05. This criterion allows for identifying any meaningful differences in performance between the direct digital PCR and duplex real-time PCR assays, contributing to the understanding of their respective sensitivities and specificities in detecting Shiga toxin-producing E. coli.

The outcome of this statistical analysis provides valuable insights into the reliability and effectiveness of the digital PCR method, informing applications in food safety testing.

### Research Results

Digital PCR (dPCR) assay was standardized and rigorously validated for the specific detection of E. coli two virulence markers (stx, eae) and a serotype-specific gene target for O157:H7. This assay was designed to enable the accurate detection of E. coli O157:H7 strains and other STEC strains and differentiation between pathogenic and non-pathogenic strains of E. coli.

Initial assay development and validation were performed using genomic DNA extracted from pure cultures of E. coli target and non-target strains. The breakdown of strains used in this study are listed in Table 3.

### Strain Characterization Using Virulence Gene-Specific PCR

Initial optimization of primer and probe concentration was performed by creating different reaction mixes, each with varying primer and probe concentrations. Tests results from this experiment showed that following optimum primer concentration for the stx (3%), eae (3%) and O157:H7 (6%) primer and for the probe stx (3%), eae (1.5%) and O157:H7 (2.8%) concentration. At the optimum primer concentration, the primer probe showed a clear separation of the positive partition cluster from the negative partition cluster.

Further results from the gradient PCR showed assay's ability to perform at an annealing temperature range of 59° C. to 67° C. Based on these results, a higher annealing temperature was selected (64° C.) for the assay. A higher annealing temperature in a PCR reaction facilitated a more specific amplification of targets. The assay showed reproducible results using the optimized conditions on the real-time PCR and dPCR instruments.

Optimization of Bacterial Cell Volume for dPCR Assay

Initial optimization of cell concentration from beef swabs was performed by creating different dilutions, each with the same primer and probe concentrations. At the optimum concentration, the plate was able to successfully separate one cell per partition and did not clog giving cleaner and easier to distinguish results.

### Hydrolysis Probe Inclusivity and Exclusivity

The assay consisted of three primer-probe sets, each demonstrated 100% inclusivity for their target genes and 100% exclusivity against non-target bacterial strains. Additionally, all pure culture test samples were tested at a higher bacterial cell concentration (10,000 dilution) and a lower dilution (100,000 dilution). Test results from bacterial cells from both concentrations showed highly specific results. These results demonstrated assay specificity at higher concentrations of non-target strains which are prone to non-specific amplification.

### Digital PCR Assay Standardization

The assay developed herein demonstrated a high sensitivity of detecting target gene within one cell. All E. coli O157:H7 strains tested using the assay were positive for all three target genes, and they showed the presence of stx and eae in the same cell in the 2D analysis. Whereas STEC strains belonging to Top 6 and non-Top 6 serogroups only showed positive for stx and eae within a single partition. The specific strain profiles used to identify O157:H7, stx, and eae are detailed in Table 8.

According to the guidelines established by FSIS, strains that test positive for both stx and eae genes are classified as pathogenic due to their potential to cause severe human illness. Conversely, strains that are negative for one or both of these genes are categorized as non-pathogenic, as they lack the complete virulence profile necessary for pathogenicity.

Assay Validation with Pure Culture Strains

Experimental validation with 18 confirmed O157:H7 strains showed consistent results, with all strains tested positive for the O157:H7-specific markers, as well as for both stx and eae in the pathogenic strains. In contrast, non-O157:H7 strains showed negative for O157:H7-specific marker, confirming the assay's ability to distinguish target strains from closely related non-target strains.

Additionally, exclusivity testing using a broader panel of 25 non-target bacterial strains tested negative for O157:H7, stx, and eae showing 100% exclusivity for the dPCR assay.

Importantly, by focusing on virulence genes rather than relying solely on serogroup-specific markers, this dPCR assay enables serogroup-independent screening of highly pathogenic E. coli strains. This is particularly valuable for identifying O157:H7 strains, which are commonly associated with severe illness and outbreaks. Additionally, it enables identifying food products contaminated with a broad range of pathogenic E. coli, which are often overlooked by regulators or private testing labs. The ability to directly detect the presence of virulent genes enhances diagnostic accuracy and facilitates rapid public health response during suspected contamination events or outbreak investigations.

Assay Validation Using Beef Swab Samples Inoculated with Pathogenic E. coli Strains

Upon performing 2D partition-based analysis (digital PCR), the results confirmed the presence of pathogenic E. coli in the inoculated samples. Pathogenic cells consistently tested positive for both eae and stx genes, appearing in the same reaction partitions. This confirmed the assay's capability to accurately detect the presence of co-expressed virulence markers within individual cells.

Assay Validation Using Beef Swabs Samples Inoculated with Non-Pathogenic E. coli Strains

To ensure the assay's specificity and its ability to differentiate between pathogenic and non-pathogenic E. coli, a parallel validation was conducted using non-pathogenic strains from the same Top 7 STEC serogroups (O157, O26, O45, O103, O111, O121, and O145). These strains were naturally lacking one of the two key virulence factors, either stx or eae. Thus, they represented stx⁻/eae⁺ or stx⁺/eae⁻ phenotypes.

The same inoculation, enrichment, and sampling protocols were applied to the non-pathogenic strains, as described above. After enrichment and centrifugation, 2D analysis was conducted to detect the presence of virulence genes.

The analysis revealed that non-pathogenic cells appeared positive for only one of the virulence markers, either O157:H7, stx, or eae, but never all within the same reaction partition. This outcome supported the assay's ability to distinguish pathogenic STEC from their non-pathogenic counterparts, ensuring that the detection of both stx and eae in the same cell is a reliable marker for STEC pathogenicity.

### Validation of Low Inoculation Testing

### Contamination Level Testing

### High Contamination Level (10 CFU/Test Portion)

All 5 replicates at the high contamination level returned positive results for E. coli detection. This yielded a POD of 1.00 (95% CI: 0.48-1.00), confirming consistent and reliable detection of the target organism at elevated concentrations.

### Fractional Positive Contamination Level (0.5 CFU/Test Portion)

Out of 20 replicates tested at the fractional level, 13 yielded positive results, producing a POD of 0.45 (95% CI: 0.25-0.66). This result reflects the expected partial detection pattern at concentrations near the assay's limit of detection. The data support the assay's utility in identifying E. coli at very low contamination levels, which are critical for food safety risk assessments and early intervention.

### Negative Control (0 CFU/Test Portion)

All 5 negative control replicates tested negative for E. coli. The resulting POD was 0.00 (95% CI: 0.00-0.48), indicating no false positives and confirming the assay's specificity under contamination-free conditions.

### Homogeneity and Sample Processing

All test portions were subjected to rigorous mixing and homogenization procedures, which were verified by consistent POD results across replicates. No significant variability was observed within contamination levels, indicating effective inoculum distribution and sampling reproducibility.

### Summary of POD Results

These results indicate that the direct digital PCR assay demonstrates high sensitivity, specificity, and robustness in detecting E. coli, including at low contamination levels and in the presence of competitive microflora. The assay is well-suited for application in beef processing environments and food safety surveillance programs.

### Discussion

### Assay Development and Specificity

The digital PCR assay was successfully developed and standardized to detect the stx, eae, and O157:H7 serotype specific gene, which are critical indicators for the presence of pathogenic E. coli strains. The assay was rigorously validated using genomic DNA extracted from pure cultures of E. coli strains representing the Top 7 Shiga toxin-producing E. coli (STEC) serogroups (O157, O26, O45, O103, O111, O121, and O145) as well as non-pathogenic strains. The results confirmed the assay's specificity, as no cross-reactivity was observed with non-target bacterial strains. This specificity is crucial for eliminating false-positives results. Further this serogroup-independent approach enabled the detection of a broad range of pathogenic strains, independent of serotype, making the dPCR assay applicable to a wider range of food safety scenarios.

Current federal regulations have a zero-tolerance policy for STEC, which necessitates a diagnostic method which is capable of detecting STEC cells at very low levels. The assay developed herein demonstrated high sensitivity, particularly in its ability to detect pathogenic strains at low contamination levels (down to 0.5 CFU/swab). Assay ability to detect such low levels of E. coli in beef is crucial and it is needed for safeguarding public health and preventing foodborne outbreaks. Regulatory agencies can implement this method for beef testing which helps them in preventing foodborne illnesses and outbreaks.

Compared to commercially available STEC testing kits, the assay developed herein has a unique ability to distinguish between pathogenic and non-pathogenic strains of E. coli. Further the assay developed has ability to differentiate stx signals from bacteriophage and eae signals originating from Enterobacteriaceae. These advantages of the diagnostic assay can facilitate better risk assessments in food production and distribution processes. This can lead to more informed decisions regarding recalls, sanitation practices, and consumer advisories, ultimately fostering greater public confidence in food safety measures. Additionally, the implementation of this diagnostic tool can significantly reduce economic losses associated with foodborne outbreaks, benefiting both producers and consumers.

As regulatory agencies increasingly emphasize the importance of rapid and accurate pathogen detection, our assay stands to play a pivotal role in promoting public health strategies aimed at mitigating the risks associated with contaminated food products.

In summary, this assay not only advances our understanding of E. coli strain differentiation but also represents a vital step towards more effective food safety practices, ultimately contributing to the reduction of foodborne illness linked to STEC contamination.

### Probe Validation

All new hydrolysis probes for the detection of E. coli O157:H7 were optimized for optimum fluorescence signal and rigorously tested using a comprehensive panel of target and non-target E. coli strains, each representing different stx gene subtypes. This optimization process of the hydrolysis probes enabled accurate separation of positive cluster from baseline cluster, which enabled clear identification of samples positive for O157:H7 specific gene, thereby minimizing the risk of cross-reactivity with non-target strains. Additionally, locked nucleotide bases were incorporated in the probe design. The addition of LNA bases improves the thermal stability of the probes, enables optimizing their annealing temperature, and thereby increasing the specificity of the TaqMan assay. This approach facilitated high annealing temperature for the probe and higher specificity and reliability of the assay.

### Assay Validation

In method validation studies, the use of a naturally contaminated matrix is generally preferred, as it provides the most accurate reflection of real-world conditions. A naturally contaminated matrix contains the target organism in its native environment, interacting with other microorganisms and existing under stresses that affect its growth, recovery, and detection. This complexity enables a more realistic assessment of a detection method's performance, particularly in terms of sensitivity and robustness. However, the use of naturally contaminated materials presents significant challenges, both practical and ethical.

In the context of the present disclosure, obtaining naturally contaminated beef samples was not feasible. While ideal from a scientific standpoint, naturally contaminated samples raise serious concerns for meat processors and suppliers, and they are very hesitant in sharing these samples. The identification of a STEC or O157:H7 positive sample could have substantial legal implications for the company, including regulatory action and potential product recalls. Additionally, any association between a beef processing company and STEC or O157:H7 contamination, even in the context of scientific research, can result in reputational damage and loss of consumer trust. Due to these risks, no commercial meat suppliers were willing to provide naturally contaminated beef matrices for use in this study. As a result, the study relied on laboratory inoculated samples using pure O157:H7 and STEC strains to simulate contamination under controlled conditions.

The physiological state of microorganisms at the time of inoculation is another important factor for assay validation studies, particularly for studies involving processed or refrigerated foods. Microorganisms in such products are often stressed due to prior exposure to environmental conditions such as refrigeration, dehydration, pH shifts, or sublethal sanitizer (e.g., chlorine, peracetic acid or lactic acid wash treatment) treatments. These stress factors can affect the physiological state of the bacteria, reducing their microbial viability, affecting their growth rate, increasing their lag phase, and or altering detection outcomes. To account for this, the inoculation procedure must reflect the conditions that closely mimic the processing environment and also factor in the conditions under which samples are shipped to the testing laboratories. For example, in raw cold-processed foods (e.g., beef), when E. coli strains accidentally contaminate beef, the finished products are swabbed with MicroTally swabs and are shipped overnight with a cold pack to an independent testing lab. Processing under refrigerated conditions and shipment of swabs under refrigerated conditions stress the microbes and slow down their growth. Therefore, to mimic the beef testing procedure the swabs after inoculation were refrigerated for 24 hours after inoculation to simulate refrigeration stress. This step ensures that the inoculated organisms closely resemble the physiologically stressed microbes found in retail or industrial samples.

### Validation of Pathogenic and Non-Pathogenic Strains

The validation of the dPCR assay using inoculated ground beef samples showed robust performance in detecting pathogenic strains. Inoculated samples with known pathogenic strains (positive for both stx and eae) consistently tested positive for these virulence markers in the same partition (double-positive partition), confirming the assay's ability to accurately identify co-expressed virulence genes within individual bacterial cells. This ability is a significant advancement over traditional PCR or a real-time PCR-based assay, which are prone to false-positive results and require culture confirmation that takes days to generate confirmation results.

In parallel, non-pathogenic strains (those possessing only one of the virulence markers stx or eae, but not both) were tested to confirm the assay's specificity for detecting STEC strains. These strains only tested positive for a single virulence marker in a partition, never for both targets in the same partition, ensuring the reliability of the dPCR assay in distinguishing between pathogenic and non-pathogenic E. coli strains. This distinction is essential, as false positives could lead to unnecessary product loss and can have major financial implications for the beef processing plant.

### Low Contamination Level Detection

An important aspect of the assay validation involved testing at various inoculation levels to evaluate its sensitivity at both high and low contamination levels. At a high contamination level (10 CFU/swab), the assay achieved a perfect detection rate (POD of 1.00), confirming its reliability at higher concentrations. More notably, at the low contamination level (0.5 CFU/swab), where swabs were inoculated in 20 replicates, the assay was able to detect E. coli O157:H7 in 45% of the replicates (POD of 0.45). A POD of 0.45 is within the expected range for fractionally inoculated samples. According to AOAC Appendix J, POD for samples inoculated at low levels must be between 0.25-0.75. These results demonstrate that our dPCR assay is capable of detecting very low levels of contamination, which is crucial for identifying food safety risk assessment and surveillance.

Further, the assay performed well for the negative control replicates (0 CFU/test portion), which tested negative in all five samples (POD of 0.00), confirming that the assay is not prone to any false positives. This high degree of accuracy ensures that the assay can reliably distinguish between contaminated and uncontaminated samples, providing confidence in its utility for routine testing in food safety programs.

### Summary and Practical Implications

In summary, the results herein confirm that the dPCR assay developed for the detection of O157:H7 and other pathogenic E. coli in beef is both sensitive and specific. The assay can detect both pathogenic and non-pathogenic strains, even in the presence of competitive microflora, at contamination levels as low as 0.5 CFU/test portion. The ability to differentiate between strains possessing both virulence markers (stx and eae) and those with only one provides a reliable marker for detecting pathogenic E. coli.

The high sensitivity and specificity makes it an ideal candidate for use in beef processing environments and for food safety surveillance programs. The assay's ability to detect pathogens at low contamination levels is particularly important for early intervention and reducing the risk of foodborne illnesses. Furthermore, by focusing on virulence genes rather than serotype-specific markers, the assay offers a serogroup-independent approach to detecting highly pathogenic E. coli strains.

In conclusion, the direct digital PCR assay represents a significant advancement in E. coli detection, offering faster, more accurate, and more reliable pathogen detection compared to traditional microbiological methods. This technology has the potential to improve food safety monitoring and contribute to a reduction in foodborne illnesses, ultimately protecting public health.

Limited work has been performed using digital PCR technology for the detection of STEC. The present disclosure builds upon previous research that utilized beef purge for pathogen inoculation and lean ground beef for inoculating the MicroTally swabs (Bosilevac, J. M., Katz, T. S., Manis, L. E., Rozier, L., & Day, M. (2025). Using Pathogenic Escherichia coli Type III Secreted Effectors espK and espV as Markers to Reduce the Risk of Potentially Enterohemorrhagic Shiga Toxin-Producing Escherichia coli in Beef. Foods, 14(3), 382. doi.org/10.3390/foods14030382; Bosilevac et al., 2025). This referenced study employed swabs prepped using a 10 mL beef purge combined with 20 g lean ground beef (fat 50, 27, and 20%). In contrast, the work disclosed herein simplified this by preparing swabs using only 20 g of ground beef (fat 20%). Beef purge is a better method for inoculating swabs as it mimics a beef processing environment; however, due to limited access to beef processing facilities near the Florida State University campus, beef purge was not able to be procured. As ground beef has a higher bacterial load compared to beef purge, the elimination of beef purge had no major effect on the assay performance.

Further, Bosilevac et al. used a relatively higher inoculation level of 50 and 5 CFU/swab, while the present disclosure focused on detecting pathogens at much lower levels, 10 and 0.5 CFU/swab. This ability of the assay to detect STEC strains at 0.5 CFU/swab reflects real-world contamination scenarios more accurately and serves to validate the assay's sensitivity. Additionally, Bosilevac et al., tested samples at 8, 15, and 24 hours of incubation. Whereas the present disclosure collected samples at 8 and 15 hours. The results show that eight-hour enrichment is sufficient for detecting STEC strains inoculated at 0.5 CFU/MicroTally swab. A shorter enrichment is preferred by the beef industry as it allows them to release the product faster. Data from samples collected following a 15-hour enrichment showed a much stronger signal, requiring a further dilution step to achieve a good separation for the 2D analysis. These results indicate that an 8-hour enrichment is optimum and extending it to 24 hours may not be necessary for accurate detection in some conditions.

Both studies targeted the key virulence genes stx and eae. However, the approach herein also incorporated specific identification of E. coli O157:H7, providing additional granularity early in the screening process. Based on input collected from a large number of beef industry food safety experts, O157:H7 is the most important target they pay attention to in their food safety plan. This ability of the assay to detect O157:H7 in the same multiplex assay facilitates quicker discrimination of high-risk strains without requiring post-PCR serotyping.

Another notable distinction for the preservation of beef enrichment samples. Bosilevac et al., encountered issues with ice crystal formation during freezing, which was mitigated by the addition of glycerol. In contrast, the standardized protocol used herein involved pelleting and directly freezing samples at −80° C., which avoided such crystallization. No degradation in assay performance was observed pre- and post-freezing, indicating the simplicity of the testing method.

In terms of results, the referenced paper, Bosilevac et al., reported optimal detection performance after 24 hours of enrichment. The present disclosure achieved equivalent or stronger signals at earlier time points (8 and 15 hours), although this required higher dilutions due to increased target concentration. This shows a more efficient detection method, reducing the total assay time needed in applied settings.

Bosilevac et al., compared mTSB and BPW for enrichment of swabs and concluded that mTSB produced superior results. The testing method herein was only validated using mTSB, and as shown in multiple previous studies, it showed consistent performance (Bosilevac et al., 2025) (Velez, F. J., Bosilevac, J. M., Salazar, G., Kaur Kapoor, H., Mishra, A., Madoroba, E., Stanford, K., Fach, P., Delannoy, S., Stephan, R., & Singh, P. (2024). Hydrolysis probe assays for the detection of pathogenic Enterohemorrhagic Escherichia coli: Multi-Country validation study. Food Research International, 196, 115105. doi.org/10.1016/j.foodres.2024.115105; Velez, F. J., Kandula, N., Blech-Hermoni, Y., Jackson, C. R., Bosilevac, J. M., & Singh, P. (2024). Digital PCR Assay for the Specific Detection and Estimation of Salmonella Contamination Levels in Poultry Rinse. Current Research in Food Science, 9, 100807. doi.org/10.1016/j.crfs.2024.100807; Valez et al., 2024). This decision avoided unnecessary variability and aligned with industry-preferred protocols.

Finally, the present disclosure included a significantly larger collection of target and non-target strains for assay validation. Previous work tested 12 eae+/stx+ strains and 24 non-infective strains: O157:H7 (n=2), O26 (n=4), O45 (n=1), O103 (n=3), O111 (n=1), O121 (n=3), and O145 (n=4) and 17 non-target bacterial strains. We included 36 eae+/stx+ strains and 79 non-pathogenic strains. The panel encompassed a broader diversity of the top seven STEC serogroups: O157:H7 (n=18), O26 (n=6), O45 (n=8), O103 (n=8), O111 (n=8), O121 (n=11), and O145 (n=5). In addition, 25 diverse non-target strains were screened to rigorously test specificity and minimize the risk of non-specific amplification.

Overall, the present disclosure presents a refined, more sensitive, and time-efficient protocol for pathogen detection in ground beef samples, supported by a more comprehensive strain validation. These improvements show a stronger practical implementation in food safety testing environments.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
PCR Calculation

| qPCR | Assay | Samples | Total |
|---|---|---|---|
| | | 13 | |
| DNA | 2 | | |
| Stx F + R | 4 | 33 | 132 |
| Stx1 P | 0.5 | 33 | 16.5 |
| Stx2 P | 0.5 | 33 | 16.5 |
| Eae F + R | 4 | 33 | 132 |
| Eae P | 0.4 | 33 | 13.2 |
| 2C F + R | 4 | 33 | 132 |
| 2C P | 0.5 | 33 | 16.5 |
| 2D F + R | 0 | 33 | 0 |
| 2D P | 0 | 33 | 0 |
| MM | 10 | 33 | 330 |
| H20 | 6.1 | 33 | 201.3 |

TABLE 2
Primer and Probe List

| Oligo | | | SEQ ID |
|---|---|---|---|
| Name | Oligo sequence | Reference | NO |
| Stx F | TTT GTY ACT GTS ACA GCW GAA GCY TTA | MLG 5C.03 | 1 |
| | CG | | |
| Stx R | CCC CAG TTC ARW GTR AGR TCM ACD TC | MLG 5C.03 | 2 |
| Stx1 | /5Cy5/CT GGA TGA T/TAO/C TCA GTG GGC | MLG 5C.03 | 3 |
| Probe | GTT CTT ATG TAA /3IAbRQSp/ | | |
| stx 2 | /5Cy5/TC GTC AGG C/TAO/A CTG TCT GAA | MLG 5C.03 | 4 |
| probe | ACT GCT CC/3IAbRQSp/ | | |
| Eae F | CAT TGA TCA GGA TTT TTC TGG TGA TA | MLG 5C.03 | 5 |
| Eae R | CTC ATG CGG AAA TAG CCG TTM | MLG 5C.03 | 6 |
| eae | /56-ROXN/AT AGT CTC GCC AGT ATT CGC | MLG 5C.03 | 7 |
| probe | CAC CAA TAC C/3IAbRQSp/ | | |
| Wang | GCG GTT TTA TTT GCA TTA GT | Wang et al., | 8 |
| Stx2C-a: | | 2002 | |
| Wang | AGT ACT CTT TTC CGG CCA CT | Wang et al., | 9 |
| Stx2C-b: | | 2002 | |
| Singh | /5HEX/TC TGT TAA T/ZEN/G CAA TGG CGG | The present | 10 |
| Stx2C- | C/3IABKFQ/ | disclosure | |
| probe | | | |
| UidA- | CCTGTAGAAACCCCAACCCG | The present | 11 |
| 48F | | disclosure | |
| UidA- | TGCCCGGCTTTCTTGTAACG | The present | 12 |
| 170R | | disclosure | |
| UidA | /56-FAM/AT TG+A +G+CA GCG TTG | The present | |
| 266-P | G/3IABKFQ/ | disclosure | |

5Cy5 refers to a 5′Cy5 fluorophore, 31AbRQSp refers to a 3′ Iowa Black Sp quencher, 56-ROXN refers to a 5′ ROX (NHS ester) — a Rhodamine fluorescent dye, 5HEX refers to a 5′ hexachlorofluorescein dye, TAO refers to an internal quencher, ZEN refers to an internal quencher, and 3IABKFQ refers a 3′ Iowa Black FQ quencher.
“Y” refers to a pyrimidine selected from either cytosine (C) or thymine (T).
“S” refers to guanine (G) or cytosine (C).
“W” refers to adenine (A) or thymine (T).
“R” refers to a purine selected from either adenine (A) or guanine (G).
“M” refers to adenine (A) or cytosine (C).
“D” refers to adenine (A), guanine (G), or cytosine (C).
“+” refers to a locked nucleic acid.

TABLE 3
Breakdown of E. coli strains, targets, and non-targets

| | Non- | | | | | | | | |
|---|---|---|---|---|---|---|---|---|---|
| Pathogenic | Pathogenic | | | | | | | | Non- |
| E. coli | E. coli | O157:H7 | O26 | O45 | O103 | O111 | O121 | O145 | Target |
| 36 | 79 | 18 | 6 | 8 | 8 | 8 | 11 | 5 | 25 |

TABLE 4
Strain Summary

| Pathogenic | Pathogenic Top | Pathogenic Non- | Non-Pathogenic |
|---|---|---|---|
| Strains | 7 Strains | Top 7 Strains | Strains |
| 36 | 21 | 15 | 79 |

TABLE 5
E. coli Pathogenic Strains

| | O157:H7 | stx | eae |
|---|---|---|---|
| E. coli O157:H7 EDL_933 | 1 | 1 | 1 |
| E. coli O157:H7 93111 | 1 | 1 | 1 |
| E. coli O157:H7 OK_1 | 1 | 1 | 1 |
| E. coli O157:H7 86_24 | 1 | 1 | 1 |
| E. coli O157:H7 493/89 | 1 | 1 | 1 |
| E. coli O157:H7 E32511/O | 1 | 1 | 1 |
| E. coli O157:H7 G5101 | 1 | 1 | 1 |
| E. coli O157:H7 932 | 1 | 1 | 1 |
| E. coli O157:H7 E0018 | 1 | 1 | 1 |
| E. coli O157:H7 H1730 | 1 | 1 | 1 |
| E. coli O157:H7 994 | 1 | 1 | 1 |
| E. coli O157:H7 K3995 | 1 | 1 | 1 |
| E. coli O157:H7 F4546 ED2 | 1 | 1 | 1 |
| E. coli O157:H7 F4492 ED | 1 | 1 | 1 |

TABLE 6
POD Results

| | | | | 95% |
|---|---|---|---|---|
| Contamination | CFU/test | No. Positive/ | | Confidence |
| Level | portion | No. Tested | POD | Interval |
| High | 10 | 5-5 | 1 | 0.48-1.00 |
| Fractional Positive | 0.5 | 9-20 | 0.45 | 0.25-0.66 |
| Negative Control | 0 | 0-5 | 0 | 0.00-0.48 |

TABLE 7
E. coli Strain Used for Assay Validation

| | O157:H7 | stx | eae |
|---|---|---|---|
| E. coli O45-1 | 0 | 0 | 1 |
| E. coli O45-3 | 0 | 0 | 1 |
| E. coli O45-4 | 0 | 0 | 1 |
| E. coli O111-1 | 0 | 1 | 1 |
| E. coli O111-3 | 0 | 0 | 1 |
| E. coli O111-4 | 0 | 0 | 1 |
| E. coli O121-2 | 0 | 1 | 1 |
| E. coli O121-3 | 0 | 1 | 1 |
| E. coli O121-4 | 0 | 1 | 1 |
| E. coli O111 EL492 | 0 | 0 | 0 |
| E. coli O111 ML699 | 0 | 0 | 0 |
| E. coli O111 WL1060 | 0 | 0 | 0 |
| E. coli O111 FSIS 75.2 | 0 | 0 | 0 |
| E. coli O26 16.2 | 0 | 0 | 0 |
| E. coli O103 33 | 0 | 0 | 0 |
| E. coli O145 56.2 | 0 | 0 | 1 |
| E. coli O103 75.2 | 0 | 0 | 0 |
| E. coli O121 75.3 | 0 | 0 | 0 |
| E. coli O26 97.1 | 0 | 0 | 1 |
| E. coli O45 151.1 | 0 | 0 | 0 |
| E. coli O45 219.1 | 0 | 0 | 1 |
| E. coli O121 219.5 | 0 | 0 | 0 |
| E. coli O145 219.7 | 0 | 0 | 0 |
| E. coli O157 221.2 | 0 | 0 | 0 |
| E. coli O45 235.1 | 0 | 0 | 0 |
| E. coli O103 302.1 | 0 | 0 | 0 |
| E. coli O145 317.2 | 0 | 0 | 0 |
| E. coli O121 508.3 | 0 | 0 | 0 |
| E. coli O103 612.1 | 0 | 0 | 1 |
| E. coli O103 621.2 | 0 | 0 | 0 |
| E. coli O45 623.3 | 0 | 0 | 0 |
| E. coli O157 645.V1 | 0 | 0 | 0 |
| E. coli O145 690.1 | 0 | 0 | 1 |
| E. coli O26 699.1 | 0 | 0 | 1 |
| E. coli O111 739.3 | 0 | 0 | 0 |
| E. coli O103 745.1 | 0 | 0 | 0 |
| E. coli O26 766.1 | 0 | 0 | 0 |
| E. coli O157 766.V1 | 0 | 0 | 0 |
| E. coli O121 785.2 | 0 | 0 | 0 |
| E. coli O103 802.1 | 0 | 0 | 0 |
| E. coli O26 859.V3 | 0 | 0 | 0 |
| E. coli O26 946.1 | 0 | 0 | 0 |
| E. coli O121 967.1 | 0 | 0 | 0 |
| E. coli O45 978.1 | 0 | 0 | 1 |
| E. coli O157 999.V1 | 0 | 0 | 0 |
| E. coli O145 170_2 | 0 | 0 | 1 |
| E. coli O121 211_1 | 0 | 0 | 1 |
| E. coli O121 256_1 | 0 | 0 | 1 |
| E. coli O157:H7 EDL_933 | 1 | 1 | 1 |
| E. coli O157:H7 93111 | 1 | 1 | 1 |
| E. coli O157:H7 OK_1 | 1 | 1 | 1 |
| E. coli O157:H7 86_24 | 1 | 1 | 1 |
| E. coli O157:H7 2886_75 | 1 | 0 | 1 |
| E. coli O157:H7 493/89 | 1 | 1 | 1 |
| E. coli O157:H7 E32511/O | 1 | 1 | 1 |
| E. coli O157:H7 G5101 | 1 | 1 | 1 |
| E. coli O55 97_3256 | 0 | 1 | 1 |
| E. coli O157:H7 932 | 1 | 1 | 1 |
| E. coli O157:H7 E0018 | 1 | 1 | 1 |
| E. coli O157:H7 H1730 | 1 | 1 | 1 |
| E. coli O157:H7 994 | 1 | 1 | 1 |
| E. coli O157:H7 F4546 | 1 | 1 | 0 |
| E. coli O157:H7 K3995 | 1 | 1 | 1 |
| E. coli O157:H7 F4546 ED2 | 1 | 1 | 1 |
| E. coli O157:H7 F4492 ED | 1 | 1 | 1 |
| E. coli O5 623.2 | 0 | 1 | 0 |
| E. coli O5 212_1 | 0 | 1 | 0 |
| E. coli O15 32167.1 | 0 | 1 | 1 |
| E. coli O74 219.4 | 0 | 1 | 0 |
| E. coli O74 388_1 | 0 | 1 | 0 |
| E. coli O84 51327 | 0 | 1 | 0 |
| E. coli O98 42128 | 0 | 1 | 0 |
| E. coli O118 12867 | 0 | 1 | 0 |
| E. coli O153 33234 | 0 | 1 | 0 |
| E. coli O172 319.1 | 0 | 1 | 1 |
| E. coli O177 285.1 | 0 | 1 | 1 |
| E. coli O177 87_1 | 0 | 1 | 1 |
| E. coli O177 92_2 | 0 | 1 | 1 |
| E. coli O182 46.1 | 0 | 1 | 1 |
| E. coli O186 12657 | 0 | 1 | 1 |
| E. coli B 674_1 | 0 | 1 | 1 |
| E. coli K 111_1 | 0 | 1 | 1 |
| E. coli N 875_1 | 0 | 1 | 1 |
| E. coli unk 86_1 | 0 | 1 | 1 |
| E. coli unk 283_1 | 0 | 1 | 1 |
| E. coli unk 759.1 | 0 | 1 | 1 |
| E. coli unk 773.1 | 0 | 1 | 1 |
| E. coli O103 519.1 | 0 | 1 | 1 |
| E. coli unk 52.2 | 0 | 1 | 1 |
| E. coli O121 81.2 | 0 | 1 | 1 |
| Acinetobacter pittii 1 | 0 | 0 | 0 |
| Acinetobacter radioresistens 28 | 0 | 0 | 0 |
| Aeromonas caviae 40 | 0 | 0 | 0 |
| Aeromonas hydrophila 89 | 0 | 0 | 0 |
| Citrobacter freundii 5 | 0 | 0 | 0 |
| Citrobacter freundii 64 | 0 | 0 | 0 |
| Enterobacter cloacae 9 | 0 | 0 | 0 |
| Enterobacter hormaechei 103 | 0 | 0 | 0 |
| Enterobacter lignolyticus 58 | 0 | 0 | 0 |
| Enterococcus faecalis 118 | 0 | 0 | 0 |
| Hafnia paralvei 26 | 0 | 0 | 0 |
| Klebsiella pneumoniae 98 | 0 | 0 | 0 |
| Morganella morganii 23 | 0 | 0 | 0 |
| Morganella morganii 61 | 0 | 0 | 0 |
| Obesumbacterium proteus 72 | 0 | 0 | 0 |
| Pantoea alhagi strain 102 | 0 | 0 | 0 |
| Proteus mirabilis strain 84 | 0 | 0 | 0 |
| Providencia alcalifaciens 69 | 0 | 0 | 0 |
| Serratia marcescens 30 | 0 | 0 | 0 |
| Skermanella aerolata 32 | 0 | 0 | 0 |
| Stenotrophomonas maltophilia 121 | 0 | 0 | 0 |
| Vibrio alginolyticus 75 | 0 | 0 | 0 |
| Vibrio fluvialis 101 | 0 | 0 | 0 |
| Vibrio parahaemolyticus 22 | 0 | 0 | 0 |
| Vibrio parahaemolyticus 100 | 0 | 0 | 0 |

What is claimed is:

1. A partition-based PCR assay comprising a primer pair selected from SEQ ID NO: 1 and SEQ ID NO: 2, SEQ ID NO: 5 and SEQ ID NO: 6, SEQ ID NO: 8 and SEQ ID NO: 9, SEQ ID NO: 11 and SEQ ID NO: 12, or a combination thereof; and a probe selected from SEQ ID NO: 3, SEQ ID NO: 4, SEQ ID NO: 7, SEQ ID NO: 10, SEQ ID NO: 13, or a combination thereof, wherein the probe comprises a modified nucleic acid and at least one detectable label.

2. The assay of claim 1, wherein the kit comprises two primer pairs selected from:
a) SEQ ID NO: 1 and SEQ ID NO: 2, and SEQ ID NO: 5 and SEQ ID NO: 6;
b) SEQ ID NO: 1 and SEQ ID NO: 2, and SEQ ID NO: 8 and SEQ ID NO: 9;
c) SEQ ID NO: 1 and SEQ ID NO: 2, and SEQ ID NO: 11 and SEQ ID NO: 12;
d) SEQ ID NO: 5 and SEQ ID NO: 6, and SEQ ID NO: 8 and SEQ ID NO: 9;
e) SEQ ID NO: 5 and SEQ ID NO: 6, and SEQ ID NO: 11 and SEQ ID NO: 12; or
f) SEQ ID NO: 8 and SEQ ID NO: 9, and SEQ ID NO: 11 and SEQ ID NO: 12

3. The assay of claim 1, wherein the kit comprises three primer pairs selected from:
a) SEQ ID NO: 1 and SEQ ID NO: 2, SEQ ID NO: 5 and SEQ ID NO: 6, and SEQ ID NO: 8 and SEQ ID NO: 9;
b) SEQ ID NO: 1 and SEQ ID NO: 2, SEQ ID NO: 5 and SEQ ID NO: 6, and SEQ ID NO: 11 and SEQ ID NO: 12;
c) SEQ ID NO: 1 and SEQ ID NO: 2, SEQ ID NO: 8 and SEQ ID NO: 9, and SEQ ID NO: 11 and SEQ ID NO: 12; or
d) SEQ ID NO: 5 and SEQ ID NO: 6, SEQ ID NO: 8 and SEQ ID NO: 9, and SEQ ID NO: 11 and SEQ ID NO: 12.

4. The assay of claim 1, wherein the kit comprises SEQ ID NO: 3, SEQ ID NO: 4, SEQ ID NO: 7, SEQ ID NO: 10, SEQ ID NO: 13, or a combination thereof.

5. The assay of claim 1, wherein the kit comprises SEQ ID NO: 3, SEQ ID NO: 4, SEQ ID NO: 7, SEQ ID NO: 10, and SEQ ID NO: 13.

6. The assay of claim 1, wherein the detectable label comprises a fluorophore, a quencher, or a combination thereof.

7. The assay of claim 1, wherein the modified nucleic acid comprises a locked nucleic acid (LNA).

8. The assay of claim 1, wherein the kit detects one or more virulent genes selected from stx, eae, or a O157:H7 serotype-specific gene.

9. The assay of claim 1, wherein the kits detects a Shiga toxin-producing Escherichia coli (E. coli) (STEC) strain.

10. The assay of claim 9, wherein the STEC strain comprises O157, O26, O45, O103, O111, O121, O145, O22, O55, O64, O86, O147, or a variant thereof.

11. The assay of claim 1, wherein the kit detects the one or more virulent genes directly within a single E. coli cell.

12. A method of detecting one or more Shiga toxin-producing E. coli (STEC) serotypes in a food product, the method comprising:
a) enriching a bacterial cell within the food product;
b) isolating the bacterial cell from the food product;
c) exposing the bacterial cell to a digital PCR component comprising at least one primer pair and at least one probe; and
d) performing a partition-based digital PCR, wherein a target nucleic acid within the bacterial cell is hybridized to at least one primer pair and at least one probe, wherein the at least one primer pair comprises SEQ ID NO: 1 and SEQ ID NO: 2, SEQ ID NO: 5 and SEQ ID NO: 6, SEQ ID NO: 8 and SEQ ID NO: 9, SEQ ID NO: 11 and SEQ ID NO: 12, or a combination thereof; and the at least one probe comprises SEQ ID NO: 3, SEQ ID NO: 4, SEQ ID NO: 7, SEQ ID NO: 10, SEQ ID NO: 13, or a combination thereof
wherein the probe comprises a modified nucleic acid and a detectable label, wherein at least one virulent gene is detected in the bacterial cell, and wherein the bacterial cell is identified as a STEC.

13. The method of claim 12, wherein the one or more STEC serotypes comprise O157, O26, O45, O103, O111, O121, O145, O22, O55, O64, O86, O147, or a variant thereof.

14. The method of claim 12, wherein the at least one virulent gene comprises stx, eae, and a O157:H7 serotype specific gene.

15. The method of claim 12, wherein the food product is enriched for no more than 8 hours.

16. The method of claim 12, wherein the method discriminates between a virulent STEC gene and an avirulent STEC gene.

17. The method of claim 12, wherein the modified nucleic acid comprises a locked nucleic acid (LNA).

18. The method of claim 12, wherein the food product comprises beef.

19. A probe comprising at least 90% sequence identity to SEQ ID NO: 10 or at least 90% sequence identity to SEQ ID NO: 13.

20. The probe of claim 19, comprising SEQ ID NO: 10 or SEQ ID NO: 13.


SEQUENCE LISTING (13 sequences)

SEQ ID NO: 1 - DNA, 29 bp, synthetic construct
  misc_feature 1..29: note = Description of sequence: Primer; Name: Stx Forward Primer
  source 1..29: mol_type = other DNA
tttgtyactgtsacagcwgaagcyttacg

SEQ ID NO: 2 - DNA, 26 bp, synthetic construct
  misc_feature 1..26: note = Description of sequence: Primer; Name: Stx Reverse Primer
  source 1..26: mol_type = other DNA
ccccagttcarwgtragrtcmacdtc

SEQ ID NO: 3 - DNA, 30 bp, synthetic construct
  misc_feature 1..30: note = Description of sequence: Probe; Name: Stx1 Probe
  source 1..30: mol_type = other DNA
  modified_base 1: mod_base = OTHER; note = cytosine bonded to a fluorescent dye, dye name: 5Cy5
  modified_base 9: mod_base = OTHER; note = n is thymine bonded at 3' carbon to internal quencher bonded to 5' carbon of cytosine; quencher name: TAO
  modified_base 30: mod_base = OTHER; note = adenine bonded to a fluorescent dye, dye name: 3IAbRQSp
ctggatgantcagtgggcgttcttatgtaa

SEQ ID NO: 4 - DNA, 26 bp, synthetic construct
  misc_feature 1..26: note = Description of sequence: Probe; Name: Stx 2 Probe
  source 1..26: mol_type = other DNA
  modified_base 1: mod_base = OTHER; note = thymine bonded to a fluorescent dye, dye name: 5Cy5
  modified_base 9: mod_base = OTHER; note = n is cytosine bonded at 3' carbon to internal quencher bonded to 5' carbon of adenine; quencher name: TAO
  modified_base 26: mod_base = OTHER; note = cytosine bonded to a fluorescent dye, dye name: 3IAbRQSp
tcgtcaggnctgtctgaaactgctcc

SEQ ID NO: 5 - DNA, 26 bp, synthetic construct
  misc_feature 1..26: note = Description of sequence: Primer; Name: Eae Forward Primer
  source 1..26: mol_type = other DNA
cattgatcaggatttttctggtgata

SEQ ID NO: 6 - DNA, 21 bp, synthetic construct
  misc_feature 1..21: note = Description of sequence: Primer; Name: Eae Reverse Primer
  source 1..21: mol_type = other DNA
ctcatgcggaaatagccgttm

SEQ ID NO: 7 - DNA, 30 bp, synthetic construct
  misc_feature 1..30: note = Description of sequence: Probe; name: Eae Probe
  source 1..30: mol_type = other DNA
  modified_base 1: mod_base = OTHER; note = adenine bonded to a Rhodamine fluorescent dye, dye name: 56-ROXN
  modified_base 30: mod_base = OTHER; note = cytosine bonded to a fluorescent dye, dye name: 3IAbRQSp
atagtctcgccagtattcgccaccaatacc

SEQ ID NO: 8 - DNA, 20 bp, synthetic construct
  misc_feature 1..20: note = Description of sequence: oligonucleotide; name: Wang et al Stx2C-a
  source 1..20: mol_type = other DNA
gcggttttatttgcattagt

SEQ ID NO: 9 - DNA, 20 bp, synthetic construct
  misc_feature 1..20: note = Description of sequence: oligonucleotide; name: Wang et al Stx2C-b
  source 1..20: mol_type = other DNA
agtactcttttccggccact

SEQ ID NO: 10 - DNA, 19 bp, synthetic construct
  misc_feature 1..19: note = Description of sequence: Probe; name: Singh Stx2C-probe
  source 1..19: mol_type = other DNA
  modified_base 1: mod_base = OTHER; note = thymine bonded to a hexachlorofluorescein dye, dye name: 5HEX
  modified_base 9: mod_base = OTHER; note = n is thymine bonded at 3' carbon to internal quencher bonded to 5' carbon of guanine; quencher name: ZEN
  modified_base 19: mod_base = OTHER; note = cytosine bonded to a fluorescent dye, dye name: 3IABkFQ
tctgttaancaatggcggc

SEQ ID NO: 11 - DNA, 20 bp, synthetic construct
  misc_feature 1..20: note = Description of sequence: oligonucleotide; name: UidA-48F
  source 1..20: mol_type = other DNA
cctgtagaaaccccaacccg

SEQ ID NO: 12 - DNA, 20 bp, synthetic construct
  misc_feature 1..20: note = Description of sequence: oligonucleotide; name: UidA-170R
  source 1..20: mol_type = other DNA
tgcccggctttcttgtaacg

SEQ ID NO: 13 - DNA, 15 bp, synthetic construct
  misc_feature 1..15: note = Description of sequence: oligonucleotide; name: UidA 266-P
  source 1..15: mol_type = other DNA
  modified_base 1: mod_base = OTHER; note = adenine bonded to a fluorescent dye, dye name: 56-FAM
  modified_base 5^6: mod_base = OTHER; note = bases connected at the 2' oxygen and 4' carbon, refered to as: locked nucleotides
  modified_base 6^7: mod_base = OTHER; note = bases connected at the 2' oxygen and 4' carbon, refered to as: locked nucleotides
  modified_base 7^8: mod_base = OTHER; note = bases connected at the 2' oxygen and 4' carbon, refered to as: locked nucleotides
  modified_base 15: mod_base = OTHER; note = guanine bonded to a fluorescent dye, dye name: 3IABkFQ
attgagcagcgttgg